SEQUENCE LISTING (9 sequences)

SEQ ID NO: 1 - protein, 256 aa, Homo sapiens
MLWQKPTAPEQAPAPARPYQGVRVKEPVKELLRRKRGHASSGAAPAPTAVVLPHQPLATYTTVGPSCLDMEGSVSAVTEEAALCAGWLSQPTPATLQPLAPWTPYTEYVPHEAVSCPYSADMYVQPVCPSYTVVGPSSVLTYASPPLITNVTTRSSATPAVGPPLEGPEHQAPLTYFPWPQPLSTLPTSTLQYQPPAPALPGPQFVQLPISIPEPVLQDMEDPRRAASSLTIDKLLLEEEDSDAYALNHTLSVEGF

SEQ ID NO: 2 - protein, 755 aa, Homo sapiens
MLDCSDYVLDSRMNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSASQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 3 - protein, 624 aa, Homo sapiens
MVHSSMGAPEIRMSKPLEAEKQGLDSPSEHTDTERNGPDTNHQNPQNKTSPFSVSPTGPSTKVGILSGLHLTFWGPGPCLSPPQIKAEDPSGDSAPAAPLPPQPAQPHLPQAQLMLTGSQLAGDIQQLLQLQQLVLVPGHHLQPPAQFLLPQAQQSQPGLLPTPNLFQLPQQTQGALLTSQPRAGLPTQAVTRPTLPDPHLSHPQPPKCLEPPSHPEEPSDLEELEQFARTFKQRRIKLGFTQGDVGLAMGKLYGNDFSQTTISRFEALNLSFKNMCKLKPLLEKWLNDAETMSVDSSLPSPNQLSSPSLGFDGLPGRRRKKRTSIETNVRFALEKSFLANQKPTSEEILLIAEQLHMEKEVIRVWFCNRRQKEKRINPCSAAPMLPSPGKPASYSPHMVTPQGGAGTLPLSQASSSLSTTVTTLSSAVGTLHPSRTAGGGGGGGGAAPPLNSIPSVTPPPPATTNSTNPSPQGSHSAIGLSGLNPSTGSTMVGLSSGLSPALMSNNPLATIQALASGGTLPLTSLDGSGNLVLGAAGAAPGSPGLVTSPLFLNHAGLPLLSTPPGVGLVSAAAAAVAASISSKSPGLSSSSSSSSSSSSSTCSETAAQTPGGPGGPEAGSKPE

SEQ ID NO: 4 - DNA, 29 bp, synthetic construct
  source 1..29: mol_type = other DNA
ttaggatccgaggagcccagtgaccttga

SEQ ID NO: 5 - DNA, 35 bp, synthetic construct
  source 1..35: mol_type = other DNA
gcaaagcttagttgattcttttttctttctggcgg

SEQ ID NO: 6 - DNA, 29 bp, synthetic construct
  source 1..29: mol_type = other DNA
ttaggatccatgctctggcaaaaacccac

SEQ ID NO: 7 - DNA, 33 bp, synthetic construct
  source 1..33: mol_type = other DNA
gcaaagcttctaaaagccttccacagagagagt

SEQ ID NO: 8 - DNA, 44 bp, synthetic construct
  source 1..44: mol_type = other DNA
aagttaaaatcacatttgaaatgcaaatggaaaagcaaggcccg

SEQ ID NO: 9 - DNA, 44 bp, synthetic construct
  source 1..44: mol_type = other DNA
cgggccttgcttttccatttgcatttcaaatgtgattttaactt

(19) United States
(12) Patent Application Publication
(10) Pub. No.: US 2025/0177412 A1
(43) Pub. Date: Jun. 5, 2025

(54) SMALL-MOLECULE INHIBITORS TARGETING THE BOB1/OCT1 INTERFACE

(71) Applicants: NANTES UNIVERSITE, Nantes (FR); INSTITUT NATIONAL DE LA SANTE ET DE LA RECHERCHE MEDICALE (INSERM), Paris (FR); ACADEMISCH MEDISCH CENTRUM, Amsterdam (NL); FEDERAL STATE BUDGETARY INSTITUTION OF SCIENCE INSTITUTE OF CYTOLOGY OF THE RUSSIAN ACADEMY OF, Saint Petersburg (RU)

(72) Inventors: Nataliya YEREMENKO, Nantes (FR); Sophie BROUARD, Sucé sur Erdre (FR); Alexey TOMILIN, Saint Petersburg (RU); Mikhail KRASAVIN, Saint Petersburg (RU); Maxim GUREEV, Saint Petersburg (RU)

(73) Assignees: NANTES UNIVERSITE, Nantes (FR); INSTITUT NATIONAL DE LA SANTE ET DE LA RECHERCHE MEDICALE (INSERM), Paris (FR); ACADEMISCH MEDISCH CENTRUM, Amsterdam (NL); FEDERAL STATE BUDGETARY INSTITUTION OF SCIENCE INSTITUTE OF CYTOLOGY OF THE RUSSIAN, Saint Petersburg (RU)

(21) Appl. No.: 18/841,721

(22) PCT Filed: Mar. 22, 2023
(86) PCT No.: PCT/EP2023/057335

§ 371 Date: Aug. 27, 2024

(30) Foreign Application Priority Data
Mar. 22, 2022 (EP) 22305339.8
Oct. 3, 2022 (RU) 2022125824

(51) Int. Cl. A61K 31/538; A61K 31/53; A61P 37/02; C07D 401/04; C07D 401/14; C07D 403/04; C07D 413/04

(52) U.S. Cl. CPC A61K 31/538; A61K 31/53; A61P 37/02; C07D 401/04; C07D 401/14; C07D 403/04; C07D 413/04

(57) ABSTRACT

Compounds of formula (I)

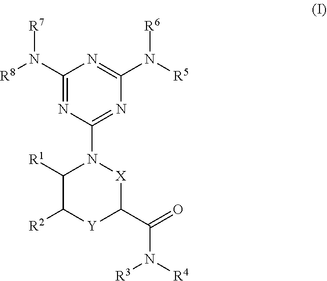

or pharmaceutically acceptable salts and/or solvates thereof. Also, the use of the compounds of formula (I) as immunomodulators and in the treatment of autoimmune diseases, transplanted organ rejection, graft-versus-host-disease and BOB1-related diseases, in which the compounds of formula (I) are administered to subjects in need thereof.

### FIELD OF INVENTION

The present invention relates to compounds of formula (I) targeting the BOB1/OCT1 interface for use as immunomodulators. The compounds of the invention are particularly useful in the treatment of autoimmune diseases, transplanted organ rejection, graft-versus-host-disease and BOB1-related diseases.

### BACKGROUND OF INVENTION

The transcriptional coactivator BOB1, which is exclusively expressed in lymphocytes, is an essential player in mounting T cell-dependent immune responses and establishment of immunological memory. BOB1 acts in cooperation with the POU-domain proteins OCT1 and OCT2. BOB1 interaction with either OCT1 or OCT2 transcription factors leads to the formation of the ternary BOB1-OCT1-DNA complexes, and activates transcription of target genes. Additionally, BOB1 aberrant expression has been linked to the development of autoimmune diseases and chronic lung transplant rejection. Conversely, the absence of functional BOB1 protein protects mice from developing such conditions.

In the setting of autoimmunity or organ transplantation, when immune response develops against self-antigens, the balance between effector and regulatory lymphocytes determines outcomes favoring the persistence of the autoimmune disease/allograft rejection or tolerance. Recent therapies for autoimmune diseases include targeting pro-inflammatory cytokines like TNF and IL-1b, inhibiting signaling pathways, blocking costimulatory molecules, or using therapeutic vaccination with regulatory T cells. For solid-organ transplantations there are no treatments except the re-transplantation. Thus, the ability to control pathogenic lymphocytic responses will offer novel therapeutic strategies to combat autoimmune diseases and transplanted organ rejection.

Based on the idea that aberrantly high expression of BOB1 promotes the formation of overactive DNA-OCT1-BOB1 ternary complex, which drives generation of pathogenic lymphocytic responses and autoimmune tissue inflammation, the Inventors developed new compounds to inhibit the biological activity of the BOB1-OCT1 axis.

The Inventors surprisingly established that the compounds of the invention can efficiently target the BOB1-interaction interface within the POU-domain of OCT1, resulting in the inhibition of BOB1/OCT1-dependent transcription. Advantageously, the Inventors demonstrated that the compounds of the invention interfere with BOB1 function and elicit a selective biological response by suppressing effector B and T lymphocytes, without affecting the regulatory capacity of B cells.

The compounds of the invention thus represent valuable potential therapeutics for the treatment of autoimmune diseases, solid transplant rejections, graft-versus-host disease and BOB1-related diseases, as well as useful tools for further study of the function of BOB1 in human health and disease.

### SUMMARY

The present invention relates to a compound of formula (I)

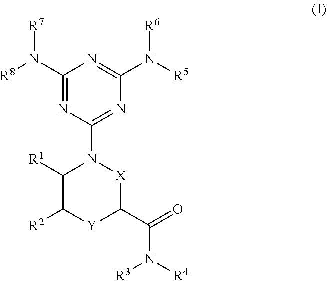

or a pharmaceutically acceptable salt and/or solvate thereof;
wherein X, Y and R¹-R⁸ are as defined hereinbelow.

The present invention further relates to a pharmaceutical composition comprising a compound according to the invention and at least one pharmaceutically acceptable carrier.

The present invention further relates to a compound according to the invention or a pharmaceutical composition according to the invention for use as a medicament. The present invention further relates to a compound according to the invention or a pharmaceutical composition according to the invention for use in the treatment of a disease selected from autoimmune diseases, transplanted organ rejection, graft-versus-host disease and BOB1-related diseases. In one embodiment, the disease to be treated is selected from rheumatoid arthritis, type 1 diabetes, multiple sclerosis, primary biliary cirrhosis, end-stage chronic respiratory diseases (such as, for example, chronic obstructive pulmonary disease (COPD), cystic fibrosis (CF) and pulmonary hypertension (PAH)), germinal center-derived lymphomas and Waldenstrom macroglobulinemia.

The present invention further relates to a process for manufacturing a compound according to the invention comprising:
(a) a step of reacting cyanuric chloride with the intracyclic NH secondary amine function of a compound of formula (A)

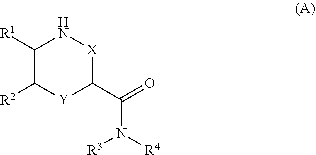

in presence of a base, thereby obtaining a monosubstituted 1,3,5-triazine of formula (B)

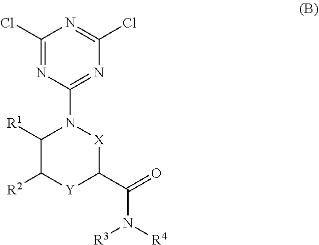

(b) a step of reacting the compound (B) with an amine of formula NHR⁵R⁶ in presence of a base, thereby obtaining a disubstituted 1,3,5-triazine; then (c) a step of reacting the resulting disubstituted 1,3,5-triazine with an amine of formula NHR⁷R⁸ in presence of a base, thereby obtaining a trisubstituted 1,3,5-triazine; or (b) a step of reacting the compound (B) with an amine of formula NHR⁷R⁸ in presence of a base, thereby obtaining a disubstituted 1,3,5-triazine; then (c) a step of reacting the resulting disubstituted 1,3,5-triazine with an amine of formula NHR⁵R⁶ in presence of a base, thereby obtaining a trisubstituted 1,3,5-triazine; and
(d) optionally, at least one step of deprotection of at least one alcohol, carboxylic acid and/or amine group present in the resulting trisubstituted 1,3,5-triazine; thereby obtaining the compound of formula (I) or the pharmaceutically acceptable salt and/or solvate thereof.

### Definitions

In the present invention, the following terms have the following meanings.

Where chemical substituents are combinations of chemical groups, the point of attachment of the substituent to the molecule is by the last chemical group recited on the right of the name of the substituent. For example, an arylalkyl substituent is linked to the rest of the molecule through the alkyl moiety and it may by represented as follows: “aryl-alkyl-”.

Unless otherwise indicated, the compounds were named using ChemDraw® Professional 15.0 (PerkinElmer).

“Alkoxy” refers to an alkyl-O— group.

“Alkyl” refers to a saturated linear or branched hydrocarbon chain, typically comprising from 1 to 12 carbon atoms, preferably from 1 to 8 carbon atoms, more preferably from 1 to 4 carbon atoms. In the present invention, alkyl groups may be monovalent or polyvalent (i.e., “alkylene” groups as defined herein are encompassed in “alkyl” definition) but alkyl groups are typically monovalent. Non-limiting examples of alkyl groups include methyl, ethyl, n-propyl, i-propyl, n-butyl, i-butyl, s-butyl and t-butyl, pentyl and its isomers (e.g., n-pentyl, iso-pentyl), and hexyl and its isomers (e.g., n-hexyl, iso-hexyl). Preferred alkyl groups include methyl, ethyl, n-propyl, i-propyl, n-butyl, s-butyl and t-butyl.

“Alkylene” refers to a divalent alkyl group. Non-limiting examples of alkylene groups include methylene, ethylene, n-propylene, i-propylene, divalent butyl, divalent pentyl and divalent hexyl. Preferred alkylene groups include methylene, ethylene, n-propylene, n-butylene and n-butylene.

“Amine” refers to derivatives of ammonia (NH₃), wherein one or more hydrogen atoms have been replaced by a substituent such as, for example, alkyl or aryl.

“Aryl” refers to a cyclic, polyunsaturated, aromatic hydrocarbyl group comprising at least one aromatic ring and comprising from 5 to 12 carbon atoms, preferably from 6 to 10 carbon atoms. Aryl groups may have a single ring (e.g., phenyl) or multiple aromatic rings fused together (e.g., naphthyl) or linked covalently. The aromatic ring may optionally include one to two additional rings (either cycloalkyl, heterocycloalkyl or heteroaryl) fused thereto. This definition of “aryl” encompasses the partially hydrogenated derivatives of the carbocyclic systems enumerated herein, as long as at least one ring is aromatic. Non-limiting examples of aryl groups include phenyl, biphenyl, biphenylenyl, 5- or 6-tetralinyl, naphthalen-1- or -2-yl, 4-, 5-, 6 or 7-indenyl, 1-2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl. A particularly preferred aryl group is phenyl.

“Cycloalkyl” refers to a cyclic monovalent alkyl group as defined herein comprising from 3 to 11 carbon atoms, preferably from 4 to 9 carbon atoms, more preferably from 5 to 7 carbon atoms. This definition of “cycloalkyl” encompasses polycyclic cycloalkyls (e.g., bicycles) and bridged cycloalkyl structures.

“Cₓ-C_{y}” or “(Cₓ-C_{y})” preceding the name of a group means that the group comprises from x to y carbon atoms, in accordance to common terminology in the chemistry field.

“Haloalkyl” refers to an alkyl group as defined herein that is substituted by at least one halogen atom, i.e., wherein at least one hydrogen atom is replaced by a halogen atom. Non-limiting examples of haloalkyl groups include fluoromethyl, difluoromethyl and trifluoromethyl.

“Heteroalkyl” refers to an alkyl group as defined herein wherein one or more carbon atoms are replaced by a heteroatom selected from oxygen, nitrogen and sulfur. In heteroalkyl groups, the heteroatoms are bound along the alkyl chain only to carbon atoms, i.e., each heteroatom is separated from any other heteroatom by at least one carbon atom. The nitrogen and sulfur heteroatoms may optionally be oxidized and the nitrogen heteroatoms may optionally be quaternized. Heteroalkyl groups may further include one or more ═O and/or ═S groups. A heteroalkyl is bound to another group or molecule only through a carbon atom, i.e., the binding atom is not selected among the heteroatoms included therein. Non-limiting examples of heteroalkyl include alkoxy, ethers and polyethers, secondary and tertiary amines and polyamines, thioethers and polythioethers, and combinations thereof.

“Heterocycloalkyl” refers to a cyclic monovalent heteroalkyl, typically comprising from 2 to 7 carbon atoms, preferably from 3 to 6 carbon atoms, more preferably from 4 to 5 carbon atoms. Heterocycloalkyl are typically 3- to 7-membered, preferably 5- or 6-membered. Heterocycloalkyl are typically monocyclic or bicyclic, preferably monocyclic. This definition encompasses polycyclic heterocycloalkyls (e.g., bicycles) and bridged heterocycloalkyl structures. Non-limiting examples of heterocycloalkyl include monovalent or divalent aziridine, pyrrolidine, piperidine, piperazine, morpholine, thiomorpholine, tetrahydrofuran and tetrahydropyran.

“Prodrug” refers to a pharmacologically acceptable derivative of a therapeutic agent (e.g., a compound of the invention) whose in vivo biotransformation product is the therapeutic agent (active drug). Prodrugs are typically characterized by increased bioavailability and are readily metabolized in vivo into the active compounds. Non-limiting examples of prodrugs include amide prodrugs and carboxylic acid ester prodrugs, in particular alkyl esters, cycloalkyl esters and aryl esters.

“Solvate” refers to molecular complex comprising a compound along with stoichiometric or sub-stoichiometric amounts of one or more molecules of one or more solvents, typically the solvent is a pharmaceutically acceptable solvent such as, for example, ethanol. The term “hydrate” refers to a solvate when the solvent is water (H₂O).

“About” is used herein to mean approximately, roughly, around, or in the region of. The term “about” preceding a figure means plus or less 10% of the value of the figure. When the term “about” is used in conjunction with a numerical range, it modifies that range by extending the boundaries above and below the numerical values set forth by 10%.

“Administration”, or a variant thereof (e.g., “administering”), means providing a therapeutic agent (e.g., a compound of the invention) alone or as part of a pharmaceutically acceptable composition, to a subject, in particular to the patient in whom/which the condition, symptom, or disease is to be treated.

“BOB1” typically refers to the human protein referenced as NP_006226.2 in the NCBI databases on Dec. 12, 2021 (corresponding to the protein of sequence SEQ ID NO: 1), or any of the predicted isoforms XP_005271650.1, XP_005271651.1, XP_006718922.1, XP_006718923.1, XP_016873421.1. In the NCBI databases (https://www.ncbi.nlm.nih.gov), the reference human POU class 2 homeobox associating factor 1 (POU2AF1) gene sequence, encoding the BOB1 protein, corresponds to NCBI Gene ID: 5450, as updated on Mar. 13, 2022. The human BOB1 gene consists of 8 exons on chromosome 11q23.1. BOB1 transcript encompasses 2875 nucleotides and encodes a 256 amino acid protein. Alternative names for BOB1 include “POU domain class 2-associating factor 1”, “B-cell-specific coactivator OBF-1”, “OCA-B”, “OCT-binding factor 1”, “OBF1”, “POU Class 2 Homeobox Associating Factor 1”, “POU Class 2 Associating Factor 1”, “BOB-1”, “OBF-1” and “OCAB”. BOB1 is a transcriptional coactivator that specifically associates with either POU2F1/OCT1 or POU2F2/OCT2. It boosts the POU2F1/OCT1 mediated promoter activity and to a lesser extent, that of POU2F2/OCT2. It has no intrinsic DNA-binding activity. It recognizes the POU domains of POU2F1/OCT1 and POU2F2/OCT2.

SEQ ID NO: 1
MLWQKPTAPEQAPAPARPYQGVRVKEPVKELLRRKRGHASSGAAP
APTAVVLPHQPLATYTTVGPSCLDMEGSVSAVTEEAALCAGWLSQ
PTPATLQPLAPWTPYTEYVPHEAVSCPYSADMYVQPVCPSYTVVG
PSSVLTYASPPLITNVTTRSSATPAVGPPLEGPEHQAPLTYFPWP
QPLSTLPTSTLQYQPPAPALPGPQFVQLPISIPEPVLQDMEDPRR
AASSLTIDKLLLEEEDSDAYALNHTLSVEGF

“BOB1-related diseases” refers to any disease in which BOB1 is aberrantly expressed.

“Comprise” or a variant thereof (e.g., “comprises”, “comprising”) is used herein according to common patent application drafting terminology. Hence, “comprise” preceded by an object and followed by a constituent means that the presence of a constituent in the object is required (typically as a component of a composition), but without excluding the presence of any further constituent(s) in the object. Moreover, any occurrence of “comprise” or a variant thereof herein also encompasses narrower expression “substantially consist of”, further narrower expression “consist of” and any variants thereof (e.g., “consists of”, “consisting of”), and may be replaced thereby, unless otherwise stated.

“Human” refers to a male or female human subject at any stage of development, including neonate, infant, juvenile, adolescent and adult.

“Kit” or “Kit of parts” are synonyms and refer to any manufacture (e.g., a package or a container) comprising a pharmaceutical composition comprising the compound according to the present invention. The kit may be promoted, distributed, or sold as a unit for performing the methods of the present invention.

“OCT1” typically refers to the human protein referenced as NP_001185712.1 in the NCBI databases on Feb. 13, 2022 (corresponding to the protein of sequence SEQ ID NO: 2), or any of the isoforms NP_002688.3, NP_001185715.1, NP_001352777.1, NP_001352778.1, or predicted isoforms XP_011507955.1, XP_011507956.1, XP_011507957.1, XP_016856997.1, XP_024303495.1. In the NCBI databases (https://www.ncbi.nlm.nih.gov), the reference human POU class 2 homeobox 1 (POU2F1) gene sequence, encoding the OCT1 protein, corresponds to NCBI Gene ID: 5451, as updated on Mar. 13, 2022. The human OCT1 gene consists of 23 exons on chromosome 1q24.2. OCT1 transcript encompasses 14007 nucleotides and encodes a 755 amino acids protein. Alternative names for OCT1 include “POU Class 2 Homeobox 1”, “OTF1”, “POU Domain, Class 2, Transcription Factor 1”, “Octamer-Binding Transcription Factor 1”, “Octamer-Binding Protein 1”, “NF-A1”, “OTF-1”, Oct-1”, “Oct-1B” and “Oct1Z”. OCT1 is a transcription factor that binds to the octamer motif (5′-ATTTGCAT-3′) and activates the promoters of the genes for some small nuclear RNAs (snRNA) and of genes such as those for histone H2B and immunoglobulins.

SEQ ID NO: 2
MLDCSDYVLDSRMNNPSETSKPSMESGDGNTGTQTNGLDFQKQPV
PVGGAISTAQAQAFLGHLHQVQLAGTSLQAAAQSLNVQSKSNEES
GDSQQPSQPSQQPSVQAAIPQTQLMLAGGQITGLTLTPAQQQLLL
QQAQAQAQLLAAAVQQHSASQQHSAAGATISASAATPMTQIPLSQ
PIQIAQDLQQLQQLQQQNLNLQQFVLVHPTTNLQPAQFIISQTPQ
GQQGLLQAQNLLTQLPQQSQANLLQSQPSITLTSQPATPTRTIAA
TPIQTLPQSQSTPKRIDTPSLEEPSDLEELEQFAKTFKQRRIKLG
FTQGDVGLAMGKLYGNDFSQTTISRFEALNLSFKNMCKLKPLLEK
WLNDAENLSSDSSLSSPSALNSPGIEGLSRRRKKRTSIETNIRVA
LEKSFLENQKPTSEEITMIADQLNMEKEVIRVWFCNRRQKEKRIN
PPSSGGTSSSPIKAIFPSPTSLVATTPSLVTSSAATTLTVSPVLP
LTSAAVTNLSVTGTSDTTSNNTATVISTAPPASSAVTSPSLSPSP
SASASTSEASSASETSTTQTTSTPLSSPLGTSQVMVTASGLQTAA
AAALQGAAQLPANASLAAMAAAAGLNPSLMAPSQFAAGGALLSLN
PGTLSGALSPALMSNSTLATIQALASGGSLPITSLDATGNLVFAN
AGGAPNIVTAPLFLNPQNLSLLTSNPVSLVSAAAASAGNSAPVAS
LHATSTSAESIQNSLFTVASASGAASTTTTASKAQ

“OCT2” typically refers to the human protein referenced as NP_001381305.1, in the NCBI databases on Dec. 20, 2021 (corresponding to the protein of sequence SEQ ID NO: 3) or any of the isoforms NP_002689.1, NP_001193954.1, NP_001193955.1, NP_001234923.1, NP_001380863.1, NP_001380864.1, NP_001380865.1, NP_001381306.1, NP_001381307.1, or predicted isoforms XP_011525343.1, XP_011525344.1, XP_011525345.2, XP_016882374.1, XP_016882373.1, XP_016882375.1, XP_016882376.1, XP_016882377.1, XP_016882378.1, XP_016882379.1, XP_016882380.1, XP_016882381.1, XP_016882383.1, XP_016882384.1, XP_016882385.1, XP_024307314.1, XP_024307315.1. In the NCBI databases (https://www.ncbi.nlm.nih.gov), the reference human POU class 2 homeobox 2 (POU2F2) gene sequence, encoding the OCT2 protein, corresponds to NCBI Gene ID: 5452, as updated on Jan. 25, 2022. The human OCT2 gene consists of 21 exons on chromosome 19q13.2. OCT2 transcript encompasses 7057 nucleotides and encodes a 624 amino acids protein. Alternative names for OCT2 include “POU Class 2 Homeobox 2”, “OTF2”, “Octamer-Binding Transcription Factor 2”, “Lymphoid-Restricted Immunoglobulin Octamer-Binding Protein NF-A2”, “POU Domain, Class 2, Transcription Factor 2”, “Octamer-Binding Protein 2”, “POU Domain Class 2, Transcription Factor 2”, “Homeobox Protein” and “OTF-2”. OCT2 is a transcription factor that specifically binds to the octamer motif (5′-ATTTGCAT-3′), a common transcription factor binding site in immunoglobulin gene promoters.

SEQ ID NO: 3
MVHSSMGAPEIRMSKPLEAEKQGLDSPSEHTDTERNGPDTNHQNP
QNKTSPFSVSPTGPSTKVGILSGLHLTFWGPGPCLSPPQIKAEDP
SGDSAPAAPLPPQPAQPHLPQAQLMLTGSQLAGDIQQLLQLQQLV
LVPGHHLQPPAQFLLPQAQQSQPGLLPTPNLFQLPQQTQGALLTS
QPRAGLPTQAVTRPTLPDPHLSHPQPPKCLEPPSHPEEPSDLEEL
EQFARTFKQRRIKLGFTQGDVGLAMGKLYGNDFSQTTISRFEALN
LSFKNMCKLKPLLEKWLNDAETMSVDSSLPSPNQLSSPSLGFDGL
PGRRRKKRTSIETNVRFALEKSFLANQKPTSEEILLIAEQLHMEK
EVIRVWFCNRRQKEKRINPCSAAPMLPSPGKPASYSPHMVTPQGG
AGTLPLSQASSSLSTTVTTLSSAVGTLHPSRTAGGGGGGGGAAPP
LNSIPSVTPPPPATTNSTNPSPQGSHSAIGLSGLNPSTGSTMVGL
SSGLSPALMSNNPLATIQALASGGTLPLTSLDGSGNLVLGAAGAA
PGSPGLVTSPLFLNHAGLPLLSTPPGVGLVSAAAAAVAASISSKS
PGLSSSSSSSSSSSSSTCSETAAQTPGGPGGPEAGSKPE

“Patient” refers to a subject who/which is awaiting the receipt of, or is receiving medical care or was/is/will be the object of a medical procedure, or is monitored for the development of the targeted disease or condition, such as, for example, autoimmune disease, transplanted organ rejection, graft-versus-host disease or BOB1-related disease.

“Pharmaceutically acceptable” means that the ingredients of a composition are compatible with each other and not deleterious to the patient to which/whom it is administered.

“Pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, e.g., FDA Office or EMA. Examples of pharmaceutically acceptable carriers include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

“Selected from” is used herein according to common patent application drafting terminology, to introduce a list of elements among which one or more item(s) is (are) selected. Any occurrence of “selected from” in the specification may be replaced by “selected from the group comprising or consisting of” and reciprocally without changing the meaning thereof.

“Subject” refers to an animal, typically a warm-blooded animal, preferably a mammal. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human. In one embodiment, the subject is a “patient” as defined herein. In one embodiment, the subject is an adult (for example a subject above the age of 18). In one embodiment, the subject is a child (for example a subject below the age of 18). In one embodiment, the subject is a male. In one embodiment, the subject is a female. In one embodiment, the subject is affected, preferably is diagnosed, with an autoimmune disease, transplanted organ rejection, graft-versus-host disease or a BOB1-related disease. In one embodiment, the subject is at risk of developing an autoimmune disease, transplanted organ rejection, graft-versus-host disease or a BOB1-related disease. Examples of risks factor include, but are not limited to, genetic predisposition, or familial history of autoimmune disease, transplanted organ rejection, graft-versus-host disease or a BOB1-related disease.

“Therapeutic agent”, “active pharmaceutical ingredient” and “active ingredient” refer to a compound for therapeutic use and relating to health. Especially, a therapeutic agent (e.g., a compound of the invention) may be indicated for treating a disease (e.g., an autoimmune disease, transplanted organ rejection, graft-versus-host disease or a BOB1-related disease). An active ingredient may also be indicated for improving the therapeutic activity of another therapeutic agent.

“Therapeutically effective amount” (in short “effective amount”) refers to the amount of a therapeutic agent (e.g., a compound of the invention) that is sufficient to achieve the desired therapeutic, prophylactic or preventative effect in the patient to which/whom it is administered, without causing significant negative or adverse side effects to said patient. A therapeutically effective amount may be administered prior to the onset of the disease, disorder, or condition, for a prophylactic or preventive action. Alternatively, or additionally, the therapeutically effective amount may be administered after initiation of the disease, disorder, or condition, for a therapeutic action.

“Treating”, “treatment” or “alleviation” refers to both therapeutic treatment and prophylactic or preventative measures, wherein the object is to prevent or slow down (lessen) the targeted pathologic condition or disorder (i.e., a “disease”). Those in need of treatment include those already with the disease as well as those prone to have the disease or those in whom the condition or disease is to be prevented. A patient is successfully “treated” for a disease if, after receiving a therapeutic amount of a therapeutic agent (e.g., a compound according the present invention), the patient shows observable and/or measurable reduction in or absence of one or more of the following: reduction in the percent of total cells that are pathogenic; and/or relief to some extent of one or more of the symptoms associated with the specific disease; reduced morbidity and mortality, and improvement in quality of life issues. The above parameters for assessing successful treatment and improvement in the disease are readily measurable by routine procedures familiar to a physician.

### DETAILED DESCRIPTION

The present invention relates to a compound of formula (I)

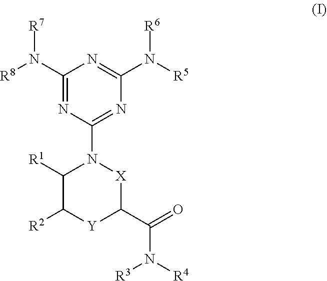

or a pharmaceutically acceptable salt and/or solvate thereof;
wherein
   X represents —(CH₂)ₙ— wherein n is 0 or 1;
   Y represents —O— or —(CH₂)ₘ— wherein m is 1 or 2;
   R¹ and R² each represents H; or R¹ and R² form together with the heterocycle to which they are attached a fused phenyl;
   R³ and R⁴ each independently represents H, alkyl, —(CH₂)ₚ—NR⁹R¹⁰, aryl or arylalkyl,
      wherein p is an integer ranging from 1 to 5,
      wherein R⁹ and R¹⁰ each independently represent H or (C₁-C₄) alkyl; or R⁹ and R¹⁰ form together with the nitrogen atom to which they are attached a heterocycloalkyl;
      wherein the aryl or arylalkyl is optionally substituted by at least one halogen, (C₁-C₄) alkyl, (C₁-C₄) haloalkyl, COOH, OH or (C₁-C₄) alkoxy;
   or R³ and R⁴ form together with the nitrogen atom to which they are attached a heterocycloalkyl;
      wherein the heterocycloalkyl is optionally substituted by at least one aryl or arylalkyl;
         wherein the aryl or arylalkyl is optionally substituted by at least one halogen, (C₁-C₄) alkyl, (C₁-C₄) haloalkyl, COOH, OH or (C₁-C₄) alkoxy;
   R⁵ represents aryl or arylalkyl;
      wherein the aryl or arylalkyl is optionally substituted by at least one halogen, (C₁-C₄) alkyl, (C₁-C₄) haloalkyl, COOH, OH or (C₁-C₄) alkoxy;
   R⁶ represents H or (C₁-C₄) alkyl; and
   R⁷ and R⁸ each independently represents H or alkyl substituted by at least one COOH or OH.

According to one embodiment, X represents a single bound, i.e., n is 0. According to one embodiment, X represents CH₂ i.e., n is 1.

According to one embodiment, Y represents —O—. According to one embodiment, Y represents CH₂ i.e., m is 1. According to one embodiment, Y represents CH₂—CH₂ i.e., m is 2.

In one embodiment, X represents a single bound and Y represents CH₂. In one embodiment, X represents a single bound and Y represents CH₂—CH₂. In one embodiment, X represents CH₂ and Y represents —O—. In one embodiment, X and Y each represents CH₂.

According to one embodiment, R¹ and R² each represents H. According to one embodiment, R¹ and R² form together with the heterocycle to which they are attached a fused phenyl.

According to one preferred embodiment, X represents CH₂, Y represents —O—, and R¹ and R² form together with the heterocycle to which they are attached a fused phenyl.

According to one embodiment, R³ and R⁴ each independently represents H, alkyl, —(CH₂)ₚ—NR⁹R¹⁰ or aryl.

According to one embodiment, R³ and R⁴ each independently represents H, alkyl or —(CH₂)ₚ—NR⁹R¹⁰; wherein p is an integer ranging from 1 to 5. In one embodiment, R³ represents —(CH₂)ₚ—NR⁹R¹⁰ wherein p is an integer ranging from 1 to 5, and R⁹ and R¹⁰ form together with the nitrogen atom to which they are attached a heterocycloalkyl such as, for example, a pyrrolidinyl (e.g., pyrrolidin-1-yl). In one embodiment, p is 3.

According to one embodiment, R⁹ and R¹⁰ form together with the nitrogen atom to which they are attached a heterocycloalkyl such as, for example, a pyrrolidinyl (e.g., pyrrolidin-1-yl).

According to one embodiment, R³ and R⁴ do not both represent H.

According to one embodiment, R³ and R⁴ do not both represent ethyl. In one embodiment, R³ and R⁴ do not both represent alkyl.

According to one embodiment, R³ represents aryl, wherein the aryl is optionally substituted by at least one halogen, (C₁-C₄) alkyl, (C₁-C₄) haloalkyl, COOH, OH or (C₁-C₄) alkoxy. In one embodiment, R³ represents aryl, wherein the aryl is optionally substituted by at least one halogen, (C₁-C₄) alkyl, (C₁-C₄) haloalkyl, COOH, OH or (C₁-C₄) alkoxy and R⁴ represents H. In one embodiment, R³ represents aryl, wherein the aryl is optionally substituted by at least one halogen, (C₁-C₄) alkyl or (C₁-C₄) haloalkyl. In one embodiment, R³ represents aryl, wherein the aryl is optionally substituted by at least one Br, Cl, F, methyl or trifluoromethyl (CF₃). In one embodiment, R³ represents aryl, wherein the aryl is optionally substituted by at least one Br, Cl, methyl or trifluoromethyl (CF₃).

According to one embodiment, R⁴ represents H.

According to one preferred embodiment, R³ represents aryl, wherein the aryl is optionally substituted by at least one halogen, (C₁-C₄) alkyl, (C₁-C₄) haloalkyl, COOH, OH or (C₁-C₄) alkoxy; and R⁴ represents H. In one preferred embodiment, R³ represents phenyl, wherein the phenyl is optionally substituted by at least one Br, Cl, methyl or trifluoromethyl (CF₃); and R⁴ represents H.

According to one embodiment, R³ and R⁴ form together with the nitrogen atom to which they are attached a heterocycloalkyl, wherein the heterocycloalkyl is optionally substituted by at least one aryl or arylalkyl. In one embodiment, R³ and R⁴ form together with the nitrogen atom to which they are attached a heterocycloalkyl, wherein the heterocycloalkyl is optionally substituted by at least one arylalkyl. In one embodiment, the heterocycloalkyl is unsubstituted. In one preferred embodiment, the heterocycloalkyl is substituted by exactly one arylalkyl. In one embodiment, the heterocycloalkyl is morpholinyl or piperidinyl. In one embodiment, the heterocycloalkyl is morpholinyl (e.g., morpholin-4-yl). In one preferred embodiment, the heterocycloalkyl is piperidinyl (e.g., piperidin-1-yl). In one embodiment, the aryl or arylalkyl is unsubstituted. In one embodiment, the aryl or arylalkyl is phenyl or benzyl. In one embodiment, the aryl is phenyl. In one embodiment, the arylalkyl is benzyl.

According to one preferred embodiment, R³ and R⁴ form together with the nitrogen atom to which they are attached a heterocycloalkyl, wherein the heterocycloalkyl is substituted by at least one aryl or arylalkyl. In one preferred embodiment, R³ and R⁴ form together with the nitrogen atom to which they are attached a heterocycloalkyl, wherein the heterocycloalkyl is substituted by at least one arylalkyl. In one preferred embodiment, the heterocycloalkyl is piperidinyl (e.g., piperidin-1-yl). In one preferred embodiment, the arylalkyl is benzyl.

According to one preferred embodiment, either: (i) R³ represents aryl, wherein the aryl is optionally substituted by at least one halogen, (C₁-C₄) alkyl, (C₁-C₄) haloalkyl, COOH, OH or (C₁-C₄) alkoxy; and R⁴ represents H; or (ii) R³ and R⁴ form together with the nitrogen atom to which they are attached a heterocycloalkyl, wherein the heterocycloalkyl is substituted by at least one aryl or arylalkyl. In one preferred embodiment, the aryl in the aryl or arylalkyl group in R³ is phenyl.

According to one embodiment, R⁵ represents arylalkyl, wherein the arylalkyl is optionally substituted by at least one halogen, (C₁-C₄) alkyl, (C₁-C₄) haloalkyl, COOH, OH or (C₁-C₄) alkoxy. In one embodiment, the arylalkyl is optionally substituted by at least one halogen, (C₁-C₄) alkyl or (C₁-C₄) haloalkyl. In one embodiment, the arylalkyl is optionally substituted by at least one Br, Cl, F, methyl or trifluoromethyl (CF₃). In one embodiment, the arylalkyl is optionally substituted by at least one F. In one embodiment, the arylalkyl is substituted by at least one F. In one particular embodiment, the arylalkyl is substituted by exactly one F. In one embodiment, the arylalkyl is substituted on the aryl part of R⁵, i.e., the substituents are not on the alkyl part thereof.

In one embodiment, R⁵ represents phenyl-(CH₂)_{q}—, wherein q is an integer ranging from 1 to 5, and wherein the phenyl is optionally substituted by at least one halogen, (C₁-C₄) alkyl, (C₁-C₄) haloalkyl, COOH, OH or (C₁-C₄) alkoxy. In one embodiment, the phenyl is optionally substituted by at least one halogen, (C₁-C₄) alkyl or (C₁-C₄) haloalkyl. In one embodiment, the phenyl is optionally substituted by at least one Br, Cl, F, methyl or trifluoromethyl (CF₃). In one embodiment, the phenyl is optionally substituted by at least one F. In one embodiment, the phenyl is substituted by at least one F. In one particular embodiment, the phenyl is substituted by exactly one F. In one embodiment, q is an integer ranging from 1 to 3. In one particular embodiment, q is 1. In one particular embodiment, q is 3.

According to one embodiment, R⁶ represents H.

In one embodiment, R⁵ represents arylalkyl, wherein the arylalkyl is optionally substituted by at least one halogen, (C₁-C₄) alkyl, (C₁-C₄) haloalkyl, COOH, OH or (C₁-C₄) alkoxy; and R⁶ represents H.

According to one embodiment, R⁷ and R⁸ each independently represents alkyl substituted by at least one COOH or OH. In one embodiment, R⁷ and R⁸ each independently represents alkyl substituted by at least one OH. In one embodiment, R⁷ and R⁸ are identical. In one embodiment, the alkyl in R⁷ and R⁸ is a (C₁-C₄) alkyl. In one preferred embodiment, the alkyl is ethyl.

According to one embodiment, R⁷ represents alkyl substituted by at least one COOH or OH. According to one embodiment, R⁷ represents alkyl substituted by at least one COOH or OH; and R⁸ represents H. In one embodiment, R⁷ represents alkyl substituted by at least one COOH. In one embodiment, the alkyl in R⁷ is a (C₁-C₄) alkyl. In one preferred embodiment, the alkyl is ethyl.

According to one embodiment, R⁸ represents H.

According to one embodiment, R⁵ represents phenyl-CH₂—, wherein the phenyl is optionally substituted by at least one halogen, (C₁-C₄) alkyl, (C₁-C₄) haloalkyl, COOH, OH or (C₁-C₄) alkoxy; and R⁷ and R⁸ each independently represents alkyl substituted by at least one OH.

According to one preferred embodiment, R⁵ represents phenyl-(CH₂)₃—, wherein the phenyl is optionally substituted by at least one halogen, (C₁-C₄) alkyl, (C₁-C₄) haloalkyl, COOH, OH or (C₁-C₄) alkoxy; R⁷ represents alkyl substituted by at least one COOH; and R⁸ represents H.

According to one embodiment, the compound of formula (I) is a compound of formula (I-A)

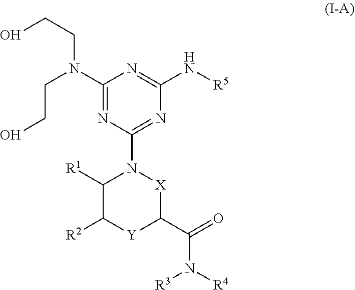

or a pharmaceutically acceptable salt and/or solvate thereof;
wherein R¹, R², R³, R⁴ and R⁵ are each independently as defined hereinabove under formula (I).

According to one embodiment, the compound of formula (I) is a compound of formula (I-B)

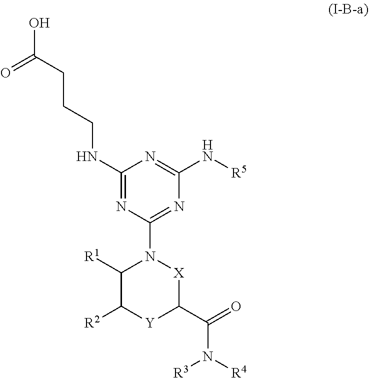

or a pharmaceutically acceptable salt and/or solvate thereof;
wherein R¹, R², R³, R⁴ and R⁵ are each independently as defined hereinabove under formula (I).

According to one preferred embodiment, the compound of formula (I) is a compound of formula (I-B-a)

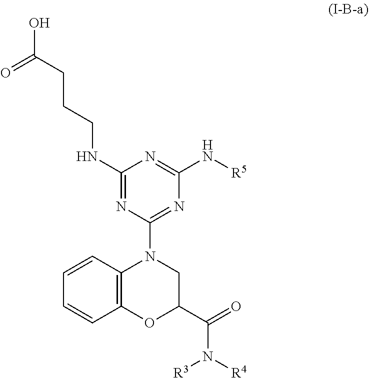

or a pharmaceutically acceptable salt and/or solvate thereof;
wherein R³, R⁴ and R⁵ are each independently as defined hereinabove under formula (I).

According to one embodiment, the compound of formula (I) is selected from the compounds of Table 1 below, and pharmaceutically acceptable salts and/or solvates thereof.

TABLE 1

| Cpd | Structure | Name |
|---|---|---|
| 001 | 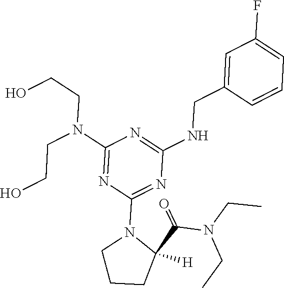 [SMILES: CCN(CC)C(=O)[C@H]1CCCN1c1nc(NCc2cccc(F)c2)nc(N(CCO)CCO)n1] | (R)-1-(4-(bis(2- hydroxyethyl)amino)- 6-((3- fluorobenzyl)amino)- 1,3,5-triazin-2-yl)- N,N- diethylpyrrolidine-2- carboxamide |
| 002 | 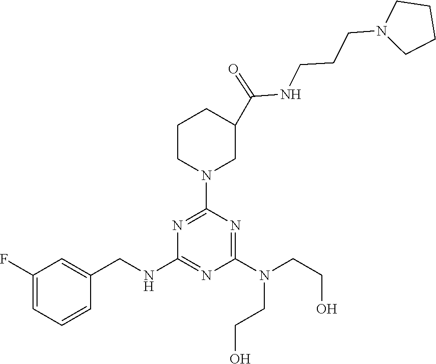 [SMILES: O=C(NCCCN1CCCC1)C1CCCN(c2nc(NCc3cccc(F)c3)nc(N(CCO)CCO)n2)C1] | 1-(4-(bis(2- hydroxyethyl)amino)- 6-((3- fluorobenzyl)amino)- 1,3,5-triazin-2-yl)-N- (3-(pyrrolidin-1- yl)propyl)piperidine- 3-carboxamide |
| 003 | 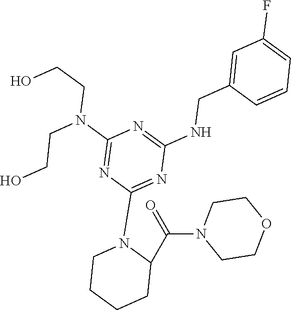 [SMILES: O=C(C1CCCCN1c1nc(NCc2cccc(F)c2)nc(N(CCO)CCO)n1)N1CCOCC1] | (1-(4-(bis(2- hydroxyethyl)amino)- 6-((3- fluorobenzyl)amino)- 1,3,5-triazin-2- yl)piperidin-2- y1)(morpholino) methanone |
| 004 | 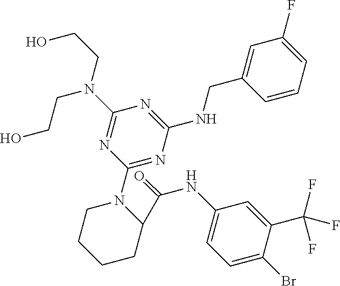 [SMILES: O=C(Nc1ccc(Br)c(C(F)(F)F)c1)C1CCCCN1c1nc(NCc2cccc(F)c2)nc(N(CCO)CCO)n1] | 1-(4-(bis(2- hydroxyethyl)amino)- 6-((3- fluorobenzyl)amino)- 1,3,5-triazin-2-y1)-N- (4-bromo-3- (trifluoromethyl) phenyl)piperidine-2- carboxamide |
| 005 | 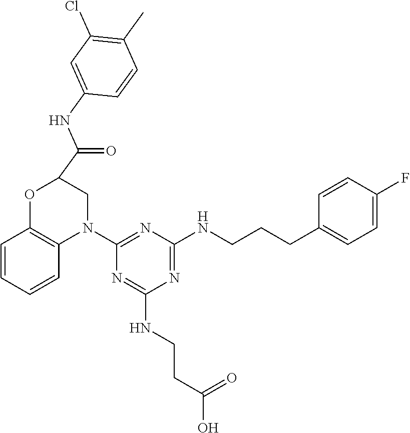 [SMILES: Cc1ccc(NC(=O)C2CN(c3nc(NCCCc4ccc(F)cc4)nc(NCCC(=O)O)n3)c3ccccc3O2)cc1Cl] | 3-((4-(2-((3-chloro-4- methylphenyl) carbamoyl)-2,3- dihydro-4H- benzo[b][1,4]oxazin- 4-y1)-6-((3-(4- fluorophenyl)propyl) amino)-1,3,5- triazin-2- yl)amino)propanoic acid |
| 006 | 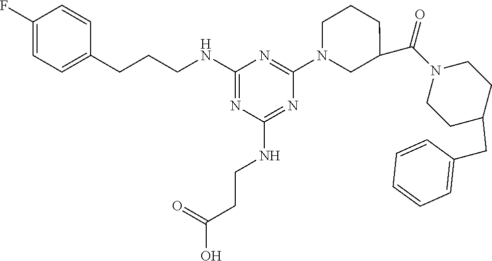 [SMILES: O=C(O)CCNc1nc(NCCCc2ccc(F)cc2)nc(N2CCCC(C(=O)N3CCC(Cc4ccccc4)CC3)C2)n1] | 3-((4-(3-(4- benzylpiperidine-1- carbonyl)piperidin-1- y1)-6-((3-(4- fluorophenyl)propyl) amino)-1,3,5- triazin-2- yl)amino)propanoic acid |

Compound 001 hereinabove named “(R)-1-(4-(bis(2-hydroxyethyl)amino)-6-((3-fluorobenzyl)amino)-1,3,5-triazin-2-yl)-N,N-diethylpyrrolidine-2-carboxamide” may alternatively be named “N-(4-(bis(2-hydroxyethyl)amino)-6-((3-fluorobenzyl)amino)-1,3,5-triazin-2-yl)-N′,N′-diethyl-L-prolinamide”.

In one embodiment, the compound of formula (I) is selected from the compounds of Table 2 below, and pharmaceutically acceptable salts and/or solvates thereof.

TABLE 2
Bis(2-hydroxyethyl)amino/3-fluorobenzylamino compounds

| Cpd | Name |
|---|---|
| 001 | (R)-1-(4-(bis(2-hydroxyethyl)amino)-6-((3-fluorobenzyl)amino)- |
| | 1,3,5-triazin-2-y1)-N,N-diethylpyrrolidine-2-carboxamide |
| 002 | 1-(4-(bis(2-hydroxyethyl)amino)-6-((3-fluorobenzyl)amino)-1,3,5- |
| | triazin-2-yl)-N-(3-(pyrrolidin-1-yl)propyl)piperidine-3-carboxamide |
| 003 | (1-(4-(bis(2-hydroxyethyl)amino)-6-((3-fluorobenzyl)amino)-1,3,5- |
| | triazin-2-y1)piperidin-2-yl)(morpholino)methanone |
| 004 | 1-(4-(bis(2-hydroxyethyl)amino)-6-((3-fluorobenzyl)amino)-1,3,5- |
| | triazin-2-y1)-N-(4-bromo-3-(trifluoromethyl)phenyl)piperidine-2- |
| | carboxamide |

According to one embodiment, the compound of formula (I) is selected from the compounds of Table 3 below, and pharmaceutically acceptable salts and/or solvates thereof.

TABLE 3
3-(4-fluorophenyl)propylamino/gamma-aminobutyric acid compounds

| Cpd | Name |
|---|---|
| 005 | 3-((4-(2-((3-chloro-4-methylphenyl)carbamoyl)-2,3-dihydro-4H- |
| | benzo[b][1,4]oxazin-4-yl)-6-((3-(4-fluorophenyl)propyl)amino)- |
| | 1,3,5-triazin-2-yl)amino)propanoic acid |
| 006 | 3-((4-(3-(4-benzylpiperidine-1-carbonyl)piperidin-1-yl)-6-((3-(4- |
| | fluorophenyl)propyl)amino)-1,3,5-triazin-2-yl)amino)propanoic acid |

According to one embodiment, the compound of formula (I) is selected from compounds 004, 005 and 006 of Table 1 hereinabove, and pharmaceutically acceptable salts and/or solvates thereof.

All references herein to a compound of the invention (e.g., “compound of formula (I)”) include references to salts, solvates, multi component complexes and liquid crystals thereof. All references herein to a compound of the invention include references to polymorphs and crystal habits thereof. All references herein to a compound of the invention include references to isotopically-labelled compounds thereof, including deuterated compounds thereof. All references herein to a compound of the invention include references to stereoisomers thereof. All herein references to a compound of the invention include references to pharmaceutically acceptable prodrugs thereof.

In particular, the compounds of the invention (e.g., a “compound of formula (I)”) may be in the form of pharmaceutically acceptable salts. According to one embodiment, the compound of the invention is a pharmaceutically acceptable salt.

Pharmaceutically acceptable salts include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. Suitable base salts are formed from bases which form non-toxic salts. Examples include the aluminum, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts. Hemisalts of acids and bases may also be formed, for example, hemisulphate and hemicalcium salts. When a compound contains an acidic group as well as a basic group the compound may also form internal salts, and such compounds are within the scope of the invention. When a compound contains a hydrogen-donating heteroatom (e.g., NH), the invention also encompasses salts and/or isomers formed by transfer of the hydrogen atom to a basic group or atom within the molecule. Pharmaceutically acceptable salts of compounds of the invention may be prepared by one or more of these methods: (i) by reacting the compound with the desired acid; (ii) by reacting the compound with the desired base; (iii) by removing an acid- or base-labile protecting group from a suitable precursor of the compound or by ring-opening a suitable cyclic precursor, e.g., a lactone or lactam, using the desired acid; and/or (iv) by converting one salt of the compound to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to almost non-ionized.

In particular, the compounds of the invention (e.g., a “compound of formula (I)”) may be in the form of pharmaceutically acceptable solvates. According to one embodiment, the compound of the invention is a pharmaceutically acceptable solvate. According to one embodiment, the compound of the invention is a pharmaceutically acceptable salt and solvate.

In particular, the compounds of the invention (e.g., a “compound of formula (I)”) may include at least one asymmetric center(s) and thus may exist as different stereoisomeric forms. Accordingly, all references to a compound of the invention include all possible stereoisomers and includes not only the racemic compounds, but the individual enantiomers and their non-racemic mixtures as well. When a compound is desired as a single enantiomer, such single enantiomer may be obtained by stereospecific synthesis, by resolution of the final product or any convenient intermediate, or by chiral chromatographic methods as each are known in the art. Resolution of the final product, an intermediate, or a starting material may be carried out by any suitable method known in the art.

The compound of the invention, as described herein, may be manufactured by synthetic methods well-known in the art.

This invention also relates to a process for manufacturing a compound of the invention, as described herein.

According to one embodiment, the process comprises:
(a) a step of reacting cyanuric chloride of formula

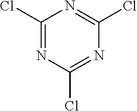

with the intracyclic NH secondary amine function of a compound of formula (A)

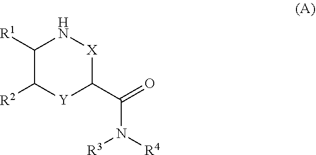

in presence of a base,
thereby obtaining a monosubstituted 1,3,5-triazine of formula (B)

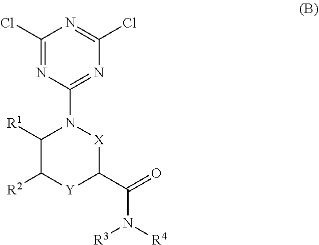

(b) a step of reacting the compound (B) with an amine of formula NHR⁵R⁶ in presence of a base, thereby obtaining a disubstituted 1,3,5-triazine; then
(c) a step of reacting the resulting disubstituted 1,3,5-triazine with an amine of formula NHR⁷R⁸ in presence of a base, thereby obtaining a trisubstituted 1,3,5-triazine;
or
(b) a step of reacting the compound (B) with an amine of formula NHR⁷R⁸ in presence of a base, thereby obtaining a disubstituted 1,3,5-triazine; then
(c) a step of reacting the resulting disubstituted 1,3,5-triazine with an amine of formula NHR⁵R⁶ in presence of a base, thereby obtaining a trisubstituted 1,3,5-triazine; and
(d) optionally, at least one step of deprotection of at least one alcohol (OH), carboxylic acid (COOH) and/or amine (NH or NH₂) group present in the resulting trisubstituted 1,3,5-triazine;
thereby obtaining the compound of formula (I) or the pharmaceutically acceptable salt and/or solvate thereof.

The process according to the invention may further comprise at least one purification and/or separation step well-known in the art.

In one embodiment, the process comprises (b) a step of reacting the compound (B) with an amine of formula NHR⁵R⁶ in presence of a base, thereby obtaining a disubstituted 1,3,5-triazine; then (c) a step of reacting the resulting disubstituted 1,3,5-triazine with an amine of formula NHR⁷R⁸ in presence of a base, thereby obtaining a trisubstituted 1,3,5-triazine.

In one embodiment, the base is a tertiary alkylamine such as, for example, N,N-Diisopropylethylamine (DIPEA).

In one embodiment, the compound of formula NHR⁶R⁷ comprises a carboxylic acid (COOH) group protected as an alkyl ester (COO-alkyl) group such as, for example, a methyl ester.

Another object of the present invention is a composition comprising or consisting essentially of a compound according to the invention, as described herein.

In one embodiment, the composition is a pharmaceutical composition and further comprises at least one pharmaceutically acceptable carrier.

Consequently, another object of the present invention is a pharmaceutical composition comprising or consisting essentially of a compound according to the invention, as described herein, and at least one pharmaceutically acceptable carrier.

As used herein, the term “consist essentially of”, with reference to a composition, pharmaceutical composition or medicament, means that the at least one compound according to the invention is the only one therapeutic agent or agent with a biologic activity within said composition, pharmaceutical composition or medicament.

Another object of the present invention is a medicament comprising or consisting essentially of a compound according to the invention, as described herein.

Another object of the invention is a kit comprising a compound according to the invention, as described herein, and instructions for use.

The present invention also relates to a compound, a composition, or a pharmaceutical composition according to the invention, as described herein, for use as a medicament.

The present invention also relates to a compound, a composition, or a pharmaceutical composition according to the invention, as described herein, for use in the treatment of an autoimmune disease, transplanted organ rejection, graft-versus-host disease or a BOB1-related disease.

The present invention further relates to a method for treating an autoimmune disease, transplanted organ rejection, graft-versus-host disease or a BOB1-related disease in a subject in need thereof, comprising administering to the subject a compound, a composition, or a pharmaceutical composition according to the invention, as described herein.

The present invention further relates to the use of a compound, a composition, or a pharmaceutical composition according to the invention, as described herein, for the manufacture of a medicament for the treatment of an autoimmune disease, transplanted organ rejection, graft-versus-host disease or a BOB1-related disease in a subject in need thereof.

The present invention also relates to the use of a compound, a composition, or a pharmaceutical composition according to the invention, as described herein, for treating an autoimmune disease, transplanted organ rejection, graft-versus-host disease or a BOB1-related disease in a subject in need thereof.

According to one embodiment, the compound is selected from the compounds of Table 1 herein. In one embodiment, the compound is selected from the compounds of Table 2 herein. In one embodiment, the compound is selected from the compounds of Table 3 herein.

Examples of autoimmune diseases include, but are not limited to, rheumatoid arthritis, type 1 diabetes, multiple sclerosis, primary biliary cirrhosis, Addison's disease, acquired immunodeficiency syndrome (AIDS), ankylosing spondylitis, anti-glomerular basement membrane disease, autoimmune hepatitis, dermatitis, Goodpasture's syndrome, granulomatosis with polyangiitis, Graves' disease, Guillain-Barre syndrome, Hashimoto's thyroiditis, hemolytic anemia, Henoch-Schonlein purpura (HSP), juvenile arthritis, juvenile myositis, Kawasaki disease, inflammatory bowel diseases (such as, for example, Crohn's disease and ulcerative colitis), polymyositis, pulmonary alveolar proteinosis, myasthenia gravis, neuromyelitis optica, Pediatric Autoimmune Neuropsychiatric Disorders Associated with Streptococcal Infections (PANDAS), psoriasis, psoriatic arthritis, Sjögren's syndrome, systemic scleroderma, systemic sclerosis, systemic lupus erythematosus, thrombocytopenic purpura (TTP), type I diabetes mellitus, uveitis, vasculitis, vitiligo, and Vogt-Koyanagi-Harada Disease.

In one embodiment, the autoimmune disease is selected from the group comprising or consisting of rheumatoid arthritis, type 1 diabetes, multiple sclerosis, and primary biliary cirrhosis.

Examples of BOB1-related diseases include, but are not limited to, end-stage chronic respiratory diseases (such as, for example, chronic obstructive pulmonary disease (COPD), cystic fibrosis (CF), pulmonary hypertension (PAH)), germinal center-derived lymphomas (Follicular lymphomas, Burkitt lymphomas, and Diffuse Large B-cell lymphomas), and Waldenstrom macroglobulinemia.

Other examples of BOB1-related diseases include, but are not limited to, solid cancers (such as, for example breast cancer, lung cancer, pancreatic cancer, colon cancer, kidney cancer, prostate cancer).

As used herein, the term “cancer” has its general meaning in the art and in particular refers to a disease caused by an uncontrolled division of abnormal cells.

Examples of solid cancers include, but are not limited to, adenoid cystic carcinoma, adrenocortical, carcinoma, AIDS-related cancers, anal cancer, appendix cancer, astrocytomas, atypical teratoid/rhabdoid tumor, basal cell carcinoma, bile duct cancer, bladder cancer, bone cancer, osteosarcoma and malignant fibrous histiocytoma, brain stem glioma, brain tumors, breast cancer, bronchial tumors, carcinoid tumors, central nervous system cancers, cervical cancer, chordoma, colon cancer, colorectal cancer, craniopharyngioma, cutaneous T cell lymphoma, embryonal tumors, endometrial cancer, ependymoblastoma, ependymoma, esophageal cancer, esthesioneuroblastoma, Ewing sarcoma family of tumors, extracranial germ cell tumor, extragonadal germ cell tumor, extrahepatic bile duct cancer, eye cancer fibrous histiocytoma of bone and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, heart cancer, hepatocellular (liver) cancer, histiocytosis, hypopharyngeal cancer, intraocular melanoma, islet cell tumors (endocrine pancreas), Kaposi sarcoma, kidney cancer, Langerhans cell histiocytosis, laryngeal cancer, lip and oral cavity cancer, liver cancer (primary), lobular carcinoma in situ (LCIS), lung cancer, male breast cancer, malignant fibrous histiocytoma of bone, medulloblastoma, medulloepithelioma, melanoma, Merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, myeloproliferative disorders, nasal cavity and paranasal sinus cancer, nasopharyngeal cancer, neuroblastoma, non-small cell lung cancer, oral cancer, oral cavity cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, pleuropulmonary blastoma and breast cancer, prostate cancer, rectal cancer, renal cell (kidney) cancer, renal pelvis and ureter, transitional cell cancer, retinoblastoma, rhabdomyosarcoma, salivary gland cancer, sarcoma, Sezary syndrome, small cell lung cancer, small intestine cancer, soft tissue sarcoma, squamous cell carcinoma, squamous neck cancer, stomach (gastric) cancer, supratentorial primitive neuroectodermal tumors, cutaneous cancer, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, cholangiocarcinoma, head and neck squamous cell carcinoma, brain glioblastoma, adrenocortical carcinoma, bladder cancer, kidney cancer, renal cancer, colorectal cancer, cervical cancer, gestational trophoblastic disease (GTD), primary peritoneal cancer.

In one embodiment, the compound according to the invention inhibits BOB1/OCT1-driven transcription. In one embodiment, the compound according to the invention reduces plasmablast differentiation. In one embodiment, the compound according to the invention abrogates B-cell activation. In one embodiment, the compound according to the invention inhibits B cell differentiation into antibody-secreting plasma cells. In one embodiment, the compound according to the invention inhibits effector B cell proliferation. In one embodiment, the compound according to the invention inhibits effector T cell proliferation. In one embodiment, the compound according to the invention suppresses memory T cell response. In one embodiment, the compound according to the invention suppresses memory Th17 cell response. In one embodiment, the compound according to the invention does not alter the regulatory function of B cells.

For use in administration to a subject, the medicament, composition, or pharmaceutical composition according to the invention, as described herein, will be formulated.

In one embodiment, the medicament, composition, or pharmaceutical composition is administered parenterally, orally, by inhalation, spray, rectally, nasally, or via an implanted reservoir.

In one embodiment, the medicament, composition, or pharmaceutical composition is administered by injection, including, without limitation, subcutaneous, intravenous, intramuscular, intra-articular, intra-synovial, intra-sternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

In one embodiment, the medicament, composition, or pharmaceutical composition is to be administered to the subject in need thereof in a therapeutically effective amount.

It will be however understood that the total daily usage of the compound, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the compound employed; the age, body weight, general health, sex and diet of the subject; the time of administration, route of administration, and rate of excretion of the specific therapeutic agent employed; the duration of the treatment; drugs used in combination or coincidental with the specific therapeutic agent employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, the dosage of the compound will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be about 0.05 to 0.5, about 0.5 to 5 or about 5 to 50 mg/kg per day. For oral administration, the compositions may be provided in the form of tablets containing from about 1.0 to 1000 milligrams of the active ingredient, particularly about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1000.0 milligrams of the active ingredient for the symptomatic adjustment of the dosage to the patient to be treated. The compounds may be administered on a regimen of 1 to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### BRIEF DESCRIPTION OF THE DRAWINGS

FIGS. 1A-C are a combination of graphs showing that compound 005 inhibits BOB1/OCT1-mediated transcription in vitro. HEC293Ea1 cells, known to express endogenous OCT1, were co-transfected with p6×W-tk-luc reporter, pEV-OBF1 (BOB1) effector, and pCMV-β-gal calibrator plasmids. Luciferase and β-galactosidase activities were measured 48 hours after transfection. The 100% luciferase activity was set as activity in presence of vehicle (10 μM DMSO). FIG. 1A shows relative luciferase activity inhibition by the compounds at the concentration 10 μM. FIG. 1B shows dose-dependent inhibition of BOB1/OCT1-mediated transcription by compound 005 in indicated concentration range (left panel). FIG. 1C shows corresponding ‘inhibitor vs normalized response with variable Hill-slope coefficient’ best-fit curve with indicated IC50 value (95% CI 4.261 to 9.422 μM). All experiments are performed at least in triplicates. Bars indicate the mean±SEM. * P<0.05, ** P<0.01, *** P<0.001, **** P<0.0001.

FIGS. 2A-D show the characterization of compound 005-OCT1 interaction in vitro. FIG. 2A shows the characterization of bacterially produced and purified His-tagged recombinant POU domain of OCT1 (POU1, 22 kDa) and BOB1 (33 kDa) on Coomassie R250-stained 12% SDS-PAGE; FIG. 2B shows electrophoretic mobility shift assay (EMSA) using the biotin-labeled PORE DNA (bio-PORE) as a probe, His-tagged BOB1 and POU1 recombinant proteins, and compound 005 (100 μM) or its vehicle (DMSO). Monomeric POU1 with the DNA, as well as supershifted by BOB1 complexes with bio-PORE are depicted; FIG. 2C shows the values of the binding constant (Kb), dissociation constant (Kd), the change of Gibb's energies (ΔG), and the number of binding sites (n) of compound 005 complex with POU1 at 298.15K. FIG. 2D shows representative results of the thermal shift assay (TSA) analysis of compound 005 complex with POU1 and, as a control, with BOB1; results are expressed as mean±SD (n=3); t-test was used for statistical analysis, n=3 (technical repeats), * P<0.05.

FIGS. 3A-Y are a combination of graphs and FACS plots showing that targeting BOB1-OCT(s) complex by compound 005 dose-dependently impairs B cells proliferation, activation and differentiation into plasmablasts in the human B cell differentiation in vitro model. Enriched memory B cells from the blood of healthy subjects (n=15) were stimulated at day 0 with anti-F(ab′)2, CD40L, CpG, and IL-2 for four days (activation phase). On day 4, cells were washed and stimulated with IL-2, IL-4, and IL-10 for additional three days (differentiation phase). Compound 005 was added daily to the cultures to a final concentration of 0 (DMSO), 5, 10, 15, 20 μM, as indicated. FIG. 3A shows representative offset histogram overlays showing median fluorescence intensity of staining of gated live B cells with cell proliferation dye (CPD), and with mAbs specific for CD27, CD38, and CD20 expression analyzed at Day 4. FIG. 3B shows corresponding summarized data for the frequency of CPD^{low} proliferating B cells analyzed at Day 4. FIG. 3C-H show corresponding summarized data for the frequency (FIG. 3C, E and G) and relative mean fluorescence intensity (MFI; FIG. 3D, F and H) of CD27⁺ (FIG. 3C-D), CD38⁺ (FIG. 3E-F), and CD20⁺ (FIG. 3G-H) B cells analyzed at Day 4. FIG. 3I shows representative offset histogram overlays showing median fluorescence intensity of staining of gated live B cells with mAbs specific for CD27, CD38, and CD20 expression at Day 7. FIG. 3J shows representative dot plots for CD20^{low}CD38⁺⁺ plasmablasts at Day 7. FIG. 3K-P show corresponding summarized data for the frequency (Fig. K, M and O) and relative mean fluorescence intensity (MFI; Fig. L, N and P) of CD27⁺ (FIG. 3K-L), CD38⁺ (FIG. 3M-N), and CD20⁺ (FIG. 3O-P) B cells analyzed at Day 7. FIG. 3Q shows corresponding summarized data for the frequency of CD20^{low}CD38⁺⁺ plasmablasts at Day 7. FIG. 3R-Y show the effect of compound 005 on the levels of IgG1 (FIG. 3R), IgG2 (FIG. 3S), IgG3 (FIG. 3T), IgG4 (FIG. 3U), IgA (FIG. 3V), IgE (FIG. 3W), IgM (FIG. 3X) and IgD (FIG. 3Y) immunoglobulin isotypes production at Day 7. Error bars are mean±SD. P values were determined with one-way ANOVA with Geisser-Greenhouse's multiple comparisons test.

FIGS. 4A-AA are a combination of heatmap plot and graphs showing expression of B-cell-related genes at day 4 and day 7 of the human B cell differentiation in vitro model in the presence of 0 μM and 10 μM of the compound 005 (n=6). Experimental conditions are as described in the FIG. 3. FIG. 4A shows unsupervised hierarchical gene clustering of 46 B-cell-related genes at day 4 and day 7 with five highlighted gene categories on the heatmap. FIG. 4B-AA show expression of the selected genes at day 4 related to proliferation (FIG. 4B-D), plasma-cell differentiation (FIG. 4E-I), immunoglobulin isotype switching (FIG. 4J-M), B-cell identity (FIG. 4N-S), and B-cell activation (FIG. 4T-AA).

FIGS. 5A-I are a combination of graphs and FACS plots showing that compound 005 does not induce B-cell apoptosis. FIG. 5A shows representative FACS plots for caspase-3 expression in B cells cultured with different concentrations of compound 005 at Day 4 and Day 7. FIG. 5B-C shows corresponding summarized data for the frequency of apoptotic B cells assessed by active caspase-3 staining at Day 4 (FIG. 5B) and Day 7 (FIG. 5C). FIG. 5D shows representative FACS plots showing the effect of compound 005 on cell apoptosis determined by active caspase-3 staining on differentiated CD20^{low}CD38⁺⁺ plasmablasts and non-differentiated CD38^{+/-} non-plasmablasts. FIG. 5E-F show corresponding summarized data (n=8) for the frequency of caspase-3⁺CD38^{+/-} non-plasmablasts (FIG. 5E) and caspase-3⁺CD20^{low}CD38⁺⁺ plasmablasts (FIG. 5F) at Day 7. FIG. 5G shows representative FACS plots of the frequency of IgD⁺CD27⁻ naïve B cells in cell cultures with specified concentrations of compound 005. FIG. 5H shows corresponding summarized data. FIG. 5I shows the effect of compound 005 on the ratio between the frequencies of IgD⁺CD27⁻ naïve B cells and CD20^{low}CD38⁺⁺ plasmablasts at Day 7. Error bars are mean±SD. P values were determined with one-way ANOVA with Geisser-Greenhouse's multiple comparisons test.

FIGS. 6A-K are a combination of graphs and FACS plots showing that targeting BOB1-OCT(s) interaction by compound 005 does not affect the suppressive capacity of B cells. B cells were kept unstimulated (B resting) or stimulated (B expanded) for 24 hours with the expansion mixture containing F(ab′)₂ anti-BCR, CpG ODN, CD40L, IL-2, and IL-21. FIG. 6A-B show the expression level of granzyme B measured as frequency (FIG. 6A) and by the MFI (FIG. 6B) in DMSO-treated (compound 005-) and 10 μM compound 005-treated resting (open circles) and expanded for 3 days (closed circles) B cells. FIG. 6C-G show resting and expanded B cells washed with fresh medium and co-cultured with autologous CPD-labelled CD4⁺CD25⁻ effector T cells for 3 days in the presence of anti-CD3/CD28 Dynabeads. compound 005 was added to the B cells only during 24-hours of activation with the expansion mixture (FIG. 6C-D), followed by daily supplementation during three days of co-culture (FIG. 6E-G). Representative offset histogram overlays showing median fluorescence intensity of staining of gated live B cells with cell proliferation dye (CPD) (FIGS. 6C, 6E and 6F). The percentage of proliferating cells is indicated for one representative donor. FIGS. 6D and 6G show corresponding summarized data for the percentage inhibition of proliferation of anti-CD3/CD28-activated CD4⁺CD25⁻ T cells co-cultured with resting (open circles) or expanded (closed circles) B cells without BOB1 inhibitor (DMSO-treated control) or in the presence of 10 μM compound 005. Bars indicate the mean±SD. ** P<0.01, *** P<0.001. FIG. 6H-J Expression values of POU2AF1 (FIG. 6H), POU2F1 (FIG. 6I), and POU2F2 (FIG. 6J) genes encoding BOB1, OCT1 and OCT2, respectively, extracted from scRNA-seq data on B cells stimulated toward regulatory (expanded) or unstimulated (rested). Data are represented with bar plots as mean+SD of non-zero values or with dot plots represented as a percentage of positive cells (FIG. 6H-J) or the average expression in each condition (FIG. 6K). P-values are adjusted to the number of expressed genes in the dataset.

FIGS. 7A-P are a combination of graphs and FACS plots showing that targeting the BOB1-OCT(s) interaction by compound 005 inhibits dose-dependently T-cell proliferation and selectively suppresses memory Th17 responses. Representative offset histogram overlays showing median fluorescence intensity of cell proliferation dye (CPD) in anti-CD3/CD28-activated CD4⁺CD25⁻ T cells cultured without BOB1 inhibitor (DMSO-treated control) or in the presence of 5, 10 or 15 μM compound 005, as indicated (FIG. 7A). The percentage of proliferating cells is indicated for one representative donor. FIG. 7B shows corresponding summarized data for the inhibition of proliferation. FIG. 7C-O show the expression of Th1-(IFNG (FIG. 7C), TNF (FIG. 7D), IL-2 (FIG. 7E) and TBX21 (FIG. 7F)), Th2-(IL-4 (FIG. 7G) and GATA3 (FIG. 7H)), Th17-(IL-17A (FIG. 7I), IL-17F (FIG. 7J), IL-21 (FIG. 7K), IL-22 (FIG. 7L) and RORC (FIG. 7M)), and Treg-(IL-10 (FIG. 7N) and FOXP3 (FIG. 7O)) associated cytokines and transcription factors during recall (memory) T cell responses in vitro. FIG. 7P shows the confirmation of qPCR data at the protein level by IL-17A intracellular staining (n=8). Each dot represents a biological replicate. Bars indicate the mean±SD. * P<0.05, ** P<0.01, **** P<0.0001.

### EXAMPLES

The present invention is further illustrated by the following examples.

### Example 1: Synthesis of the Compounds

### Materials and Methods

All reagents were purchased from commercial sources and used as received. NMR spectroscopic data were recorded with a 400 MHz spectrometer (400.13 MHz for ¹H and 100.61 MHz for ¹³C) in CDCl₃ and in DMSO-d₆ and were referenced to residual solvent proton signals (δ_{H}=7.26 and δ_{H}=2.50, respectively) and solvent carbon signals (δ_{C}=77.0 and δ_{C}=39.5, respectively). All chemical shifts are reported in parts per million (ppm). Abbreviations used in the description of resonances are as follows: s (singlet), d (doublet), t (triplet), q, (quartet), br (broad), m (multiplet). Coupling constants (J) are quoted to the nearest 0.1 Hz. Mass spectra were recorded with a HRMS-ESI-qTOF spectrometer (electrospray ionization mode). Melting points were determined with a melting point apparatus Stuart SMP 50 in open capillary tubes. Flash column chromatography was performed using silica gel Merk grade 60 (0.040-0.063 mm) 230-400 mesh (isocratic or gradient elution as indicated).

### Synthetic Method

Example compounds 001-006 were prepared starting from cyanuric chloride as shown on Scheme 1 below for representative compound 005.

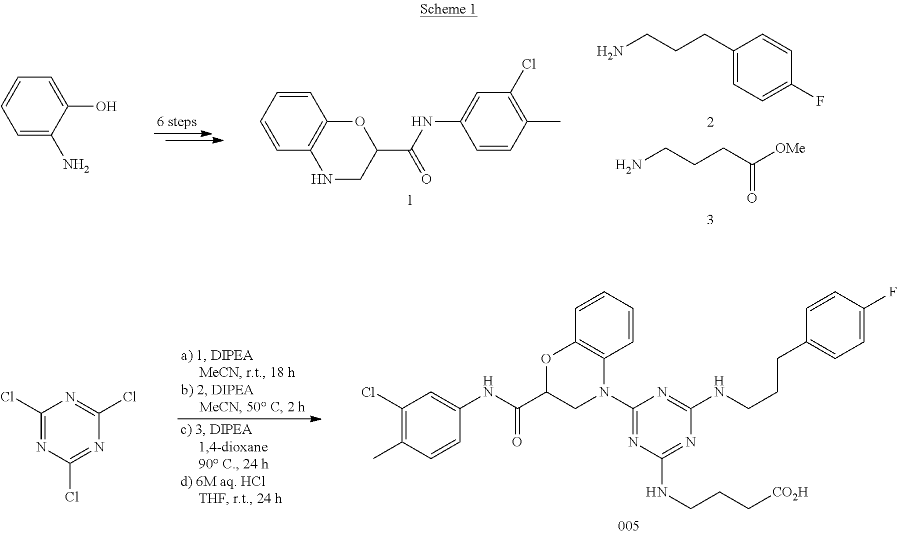 [SMILES: Nc1ccccc1O ; Cc1ccc(NC(=O)C2CNc3ccccc3O2)cc1Cl ; NCCCc1ccc(F)cc1 ; COC(=O)CCCN ; Clc1nc(Cl)nc(Cl)n1 ; Cc1ccc(NC(=O)C2CN(c3nc(NCCCC(=O)O)nc(NCCCc4ccc(F)cc4)n3)c3ccccc3O2)cc1Cl]

The synthesis of 005 required initial elaboration of benzoxazine amine 1 and the use of commercially available reagents 2 and 3. Sequential nucleophilic displacement of the three chlorine atoms in cyanuric chloride required increasingly forcing conditions. Acidic hydrolysis of the ester group in the final step provided the target compound 005.

Example compounds 001-004 and 006 were prepared following a similar method, as detailed hereinafter.

### Results

### Example Compound 005: 3-((4-(2-((3-chloro-4-methylphenyl)carbamoyl)-2,3-dihydro-4H-benzo[b][1,4]oxazin-4-yl)-6-((3-(4-fluorophenyl)propyl)amino)-1,3,5-triazin-2-yl)amino)propanoic Acid (005)

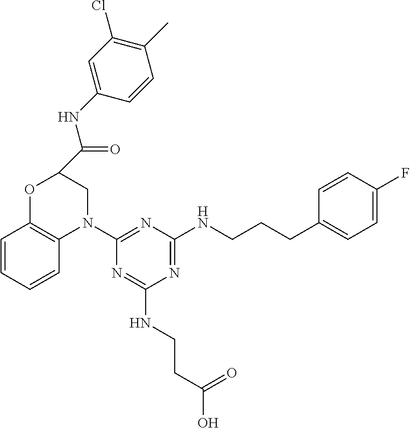 [SMILES: Cc1ccc(NC(=O)C2CN(c3nc(NCCCc4ccc(F)cc4)nc(NCCC(=O)O)n3)c3ccccc3O2)cc1Cl]

To a stirred mixture of 2-aminophenol (4.0 g, 37 mmol), NaOAc·3H₂O (14.2 g, 105 mmol) and 40% (v/v) aq. ethanol (100 mL) was added 2-nitrobenzenesulfonyl chloride (7.5 g, 35 mmol) in small portions during 20 min, then the mixture was brought to reflux, cooled, diluted with water (200 mL), acidified with conc. HCl. The dark precipitate was filtered off, washed with water and treated with boiling ethanol (50 mL). An insoluble solid was discharged, the hot filtrate was diluted with water (50 mL). After cooling overnight, the precipitate was filtered off, washed with water and dried to afford crude N-(2-hydroxyphenyl)-2-nitrobenzenesulfonamide (6.0 g, 57%) as a deep purple solid.

A mixture of crude N-(2-hydroxyphenyl)-2-nitrobenzenesulfonamide (5.9 g, 20 mmol), methyl 2,3-dibromopropionate (5.5 g, 2.2 mmol), K₂CO₃ (6.9 g, 50 mmol) and DMF (70 mL) was stirred vigorously at 50° C. overnight, cooled, taken into water (500 mL) and extracted with DCM (3×70 mL). The combined DCM phase was washed successively with water (3×70 mL) dried over K₂CO₃ and concentrated in vacuo to give methyl 4-((2-nitrophenyl)sulfonyl)-3,4-dihydro-2H-benzo[b][1,4]oxazine-2-carboxylate (4.91 g, 65%) as a red oil which was used in the next step without any purification. ¹H NMR (400 MHz, CDCl₃) δ 7.94 (d, J=7.8 Hz, 1H), 7.81-7.66 (m, 3H), 7.51 (dd, J=8.3, 1.0 Hz, 1H), 7.21-7.12 (m, 1H), 7.05 (dd, J=8.2, 1.2 Hz, 1H), 7.01-6.92 (m, 1H), 4.67 (dd, J=8.4, 3.0 Hz, 1H), 4.52 (dd, J=14.4, 3.0 Hz, 1H), 4.56-4.45 (m, 1H), 3.86 (s, 3H), 3.80-3.72 (dd, J=8.4, 3.0 Hz, 1H).

The ester thus obtained (4.8 g, 12.7 mmol) was dissolved in EtOH (70 mL) at 50° C. and treated with 40 mL aq. KOH (1.9 g, 34 mmol) during 5 min under stirring. The resulting clear solution was diluted with water (100 mL) and acidified with conc. HCl to pH<2. The yellow precipitate was filtered off, washed with water and recrystallized from aq. EtOH to give 4-((2-nitrophenyl)sulfonyl)-3,4-dihydro-2H-benzo[b][1,4]oxazine-2-carboxylic acid (3.93 g, 85%) as a pale yellow powder. ¹H NMR (400 MHz, acetone-d₆) δ 11.7 (br. s., 1H), 8.07-7.96 (m, 3H), 7.94-7.85 (m, 1H), 7.44 (dd, J=8.3, 1.4 Hz, 1H), 7.20-7.11 (m, 1H), 7.02 (dd, J=8.2, 1.4 Hz, 1H), 6.95 (ddd, J=8.6, 7.4, 1.5 Hz, 1H), 4.80 (dd, J=7.5, 3.1 Hz, 1H), 4.45 (dd, J=14.2, 3.2 Hz, 1H), 4.02 (dd, J=14.2, 7.5 Hz, 1H).

To a suspension of that acid (3.8 g, 10.4 mmol) in DCM (10 mL) thionyl chloride (1 mL) and 1 drop DMF were added, and the resulting mixture was stirred under reflux to from a clear solution and then additionally for 20 min, then concentrated in vacuo to obtain crude 4-((2-nitrophenyl)sulfonyl)-3,4-dihydro-2H-benzo[b][1,4]oxazine-2-carbonyl chloride as an orange oil, which was used in the next step without any purification.

To a stirred solution of 3-chloro-4-methylaniline (780 mg, 5.5 mmol) and NMM (1 mL) in DCM (30 mL) was added the solution of above acyl chloride (1.9 g, 5.0 mmol) in DCM (15 mL) during 2 min. The stirring was continued for 30 min, then the resulting mixture was washed with 5% HCl (2×20 mL), water, 5% NaOH (10 mL), water again, dried over K₂CO₃ and concentrated in vacuo. The residue was crystallized from Et₂O-DCM to give N-(3-chloro-4-methylphenyl)-4-((2-nitrophenyl)sulfonyl)-3,4-dihydro-2H-benzo[b][1,4]oxazine-2-carboxamide (2.0 g, 75%) as a pale yellow solid. ¹H NMR (400 MHz, CDCl₃) δ 8.28 (s, 1H), 7.70 (d, J=2.0 Hz, 1H), 7.36 (dd, J=8.2, 2.1 Hz, 1H), 7.20 (d, J=8.2 Hz, 1H), 6.99 (dd, J=8.0, 1.1 Hz, 1H), 6.88 (td, J=7.6, 1.2 Hz, 1H), 6.78 (td, J=7.8, 1.3 Hz, 1H), 6.71 (dd, J=7.8, 1.3 Hz, 1H), 4.78 (dd, J=7.3, 2.9 Hz, 1H), 3.80 (dd, J=11.9, 2.9 Hz, 1H), 3.54 (dd, J=11.9, 7.3 Hz, 1H), 2.36 (s, 3H).

A mixture of the previous amide (1.90 g, 3.9 mmol), thiophenol (1.29 g, 11.7 mmol), K₂CO₃ (2.29 g, 19.5 mmol) and DMF (20 mL) was stirred at 40° C. for 5 h in a sealed tube, then poured into 3% aq. NaOH (60 mL) and extracted with DCM (3×20 mL). The combined organic phases were washed with 3% aq. NaOH, water, and the product was extracted with 5% HCl (3×20 mL). The aqueous combined phases were washed with DCM and alkalized with 10% aq. NaOH. The product was extracted with DCM (3×20 mL), washed with water, dried over MgSO₄, concentrated in vacuo to obtain crude N-(3-chloro-4-methylphenyl)-3,4-dihydro-2H-benzo[b][1,4]oxazine-2-carboxamide 1, which was recrystallized from Et₂O-DCM to give pure compound (907 mg, 77%) as a beige solid. ¹H NMR (400 MHz, CDCl₃) δ 8.28 (br. s, 1H), 7.70 (d, J=2.0 Hz, 1H), 7.36 (dd, J=8.2, 2.1 Hz, 1H), 7.20 (d, J=8.2 Hz, 1H), 6.99 (dd, J=8.0, 1.1 Hz, 1H), 6.88 (td, J=7.6, 1.2 Hz, 1H), 6.78 (td, J=7.8, 1.3 Hz, 1H), 6.71 (dd, J=7.8, 1.3 Hz, 1H), 4.78 (dd, J=7.3, 2.9 Hz, 1H), 3.80 (dd, J=11.9, 2.9 Hz, 1H), 3.54 (dd, J=11.9, 7.3 Hz, 1H), 3.3 (br. s, 1H), 2.36 (s, 3H).

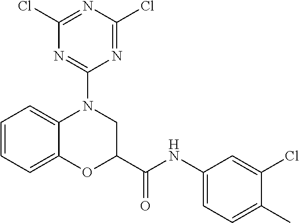 [SMILES: Cc1ccc(NC(=O)C2CN(c3nc(Cl)nc(Cl)n3)c3ccccc3O2)cc1Cl]

A mixture of cyanuric chloride (213 mg, 1.15 mmol), the previous amine 1 (350 mg, 1.15 mmol), DIPEA (164 mg, 127 mmol) and acetonitrile (20 mL) was stirred overnight at RT. The solvent was evaporated in vacuo, the residue dissolved in DCM (50 mL), resulting solution washed with satd. NaHCO₃, dried over MgSO₄, and concentrated in vacuo. The product was isolated by column chromatography on silica gel eluting with EtOAc-DCM (1:1) to afford N-(3-chloro-4-methylphenyl)-4-(4,6-dichloro-1,3,5-triazin-2-yl)-3,4-dihydro-2H-benzo[b][1,4]oxazine-2-carboxamide (301 mg, 58%) as a beige solid. ¹H NMR (400 MHz, CDCl₃) δ 8.21 (br. s, 1H), 8.03 (dd, J=8.3, 1.1 Hz, 1H), 7.63 (d, J=2.0 Hz, 1H), 7.34 (dd, J=8.2, 2.0 Hz, 1H), 7.27-7.08 (m, 4H), 5.00 (dd, J=13.6, 3.2 Hz, 1H), 4.92 (dd, J=7.7, 3.2 Hz, 1H), 4.18 (dd, J=13.7, 7.7 Hz, 1H), 2.36 (s, 3H).

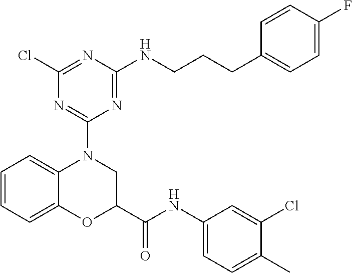 [SMILES: Cc1ccc(NC(=O)C2CN(c3nc(Cl)nc(NCCCc4ccc(F)cc4)n3)c3ccccc3O2)cc1Cl]

A solution of the previous compound N-(3-chloro-4-methylphenyl)-4-(4,6-dichloro-1,3,5-triazin-2-yl)-3,4-dihydro-2H-benzo[b][1,4]oxazine-2-carboxamide (285 mg, 0.633 mmol), 3-(4-fluorophenyl) propan-1-amine 2 (107 mg, 699 mmol) and DIPEA (91 mg, 71 mmol) in acetonitrile (25 mL) was stirred at 50° C. for 2 h. The solvent was evaporated in vacuo, the residue was triturated with satd. aq. NaHCO₃ solution, the white solid product was filtered off, washed with water and a little Et₂O, and dried on air to afford 320 mg (89%) N-(3-chloro-4-methylphenyl)-4-(4-chloro-6-((3-(4-fluorophenyl)propyl)amino)-1,3,5-triazin-2-yl)-3,4-dihydro-2H-benzo[b][1,4]oxazine-2-carboxamide ¹H NMR (400 MHz, DMSO-d₆) δ 10.23 (br. s, 1H), 8.25-8.05 (m, 1H), 8.17 (d, J=45.8 Hz, 1H), 7.88 (br. t, 1H), 7.68-7.63 (m, 1H), 7.49-6.80 (m, 5H), 5.10-4.91 (m, 1H), 4.75-4.15 (br. m, 2H), 3.29-3.06 (m, 2H), 2.66-2.51 (m, 3H), 2.25 and 2.27 (s, 3H), 1.77 (m 2H). A ca. 3:1 mixture of two rotamers.

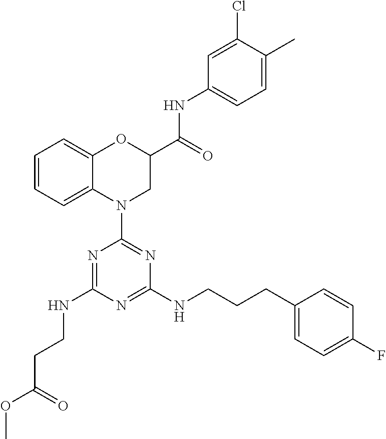 [SMILES: COC(=O)CCNc1nc(NCCCc2ccc(F)cc2)nc(N2CC(C(=O)Nc3ccc(C)c(Cl)c3)Oc3ccccc32)n1]

Methyl 3-((4-(2-((3-chloro-4-methylphenyl)carbamoyl)-2,3-dihydro-4H-benzo[b][1,4]oxazin-4-yl)-6-((3-(4-fluorophenyl)propyl)amino)-1,3,5-triazin-2-yl)amino)propanoate. A mixture of (290 mg, 0.511 mmol), β-alanine methyl ester hydrochloride (143 mg, 1.02 mmol) DIPEA (199 mg, 1.54 mmol) in 1,4-dioxane (10 mL) was heated at 90° C. for 24 h. A satd. NaHCO₃ solution (30 mL) was added and the resulting suspension was extracted with DCM (3×30 mL). The combined organic extracts were washed with water (75 mL) and dried over solid anhydrous MgSO₄. After filtration, the solvent was removed under reduced pressure and the residue was purified by flash column chromatography eluting with DCM-EtOAc (1:1) to give the title compound (210 mg, 65%) as a white amorphous solid. ¹H NMR (400 MHz, DMSO-d₆) δ 10.16 (br. s, 1H), 8.07 (br. s, 1H), 7.74 (br. s, 1H), 7.43 (br. s, 1H), 7.33-7.15 (m, 3H), 7.15-6.69 (m, 7H), 5.04-3.74 (br. m, 3H), 3.59 (s, 3H), 3.52-3.37 (m, 2H), 3.25-3.10 (m, 2H), 2.56 (m, 4H), 2.27 (s, 3H), 1.76 (br. s, 2H).

3-((4-(2-((3-chloro-4-methylphenyl)carbamoyl)-2,3-dihydro-4H-benzo[b][1,4]oxazin-4-yl)-6-((3-(4-fluorophenyl)propyl)amino)-1,3,5-triazin-2-yl)amino)propanoic acid (005). A mixture of the previous methyl ester (200 mg, 0.315 mmol), 6 N aq. HCl (3 mL) and THF (5 ml) was stirred at RT for 24 h. After this time, the pH of the clear solution was adjusted to ˜5 by addition of 25% aq. NH₃. The mixture was stirred vigorously for 1 h, the precipitate formed was filtered off, washed with water and dried to give 110 mg (56%) of the title compound (005) as a white amorphous solid. ¹H NMR (400 MHz, DMSO-d₆) δ 12.15 (br. s, 1H), 10.16 (br. s, 1H), 8.2-6.5 (br. m, 15H), 5.21-3.55 (m, 6H), 2.27 (s, 3H), 1.77 (br. s, 2H). A part of signals overlapped with water. HRMS (ESI-TOF): [C₃₁H₃₁ClFN₇O₄+H]⁺. found 620.2191. calcd. 620.2183.

### Example Compound 002: 1-(4-(bis(2-hydroxyethyl)amino)-6-((3-fluorobenzyl)amino)-1,3,5-triazin-2-yl)-N-(3-(pyrrolidin-1-yl)propyl)piperidine-3-carboxamide (002)

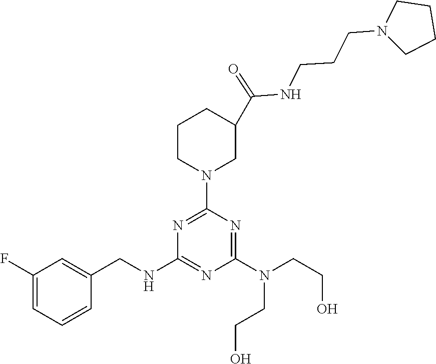 [SMILES: O=C(NCCCN1CCCC1)C1CCCN(c2nc(NCc3cccc(F)c3)nc(N(CCO)CCO)n2)C1]

N-(3-(pyrrolidin-1-yl)propyl)piperidine-3-carboxamide was prepared from (±)-N-Boc-nipecotic acid and 1-(3-aminopropyl) pyrrolidine by standard mixed anhydride procedure (IBCF, NMM, DCM; column chromatography) followed by Boc-cleavage (TFA, DCM; then aq. NaHCO₃) and used in the next step without any purification.

N-(4-chloro-6-(3-fluorobenzylamino)-1,3,5-triazin-2-yl) diethanolamine. To an ice-cooled suspension of cyanuric chloride (1.85 g, 10.0 mmol) in DCM (50 ml) was added the mixture of 3-fluorobenzylamine (1.25 g, 10.0 mmol) and DIPEA (1.42 g, 11.0 mmol) dissolved in DCM (25 mL) during 15 min. The resulting mixture was stirred under ice-cooling for 2 h and then allowed to warm up to RT. Afterwards an emulsion of diethanolamine (1.16 g, 11.0 mmol) in DCM (25 mL) was added and the mixture was stirred at RT for 20 h, the washed rapidly with satd. NaHCO₃ solution (30 mL), then water (partial crystallization of the product), and concentrated under reduced pressure. The white product was crystallized using DCM-Et₂O mixture, filtered off and dried on air to give 2.20 g (65%) of N-(4-chloro-6-(3-fluorobenzylamino)-1,3,5-triazin-2-yl) diethanolamine. ¹H NMR (400 MHz, DMSO-d₆, 100° C.) δ 7.95 (br. s, 1H), 7.35 (td, J=7.9, 6.2 Hz, 1H), 7.16 (d, J=7.7 Hz, 1H), 7.10 (br. d, J=10.2 Hz, 1H), 7.02 (td, J=8.5, 2.3 Hz, 1H), 4.48 (d, J=6.3 Hz, 2H), 4.38 (br. s, 2H), 3.70-3.52 (m, 8H).

1-(4-(bis(2-hydroxyethyl)amino)-6-((3-fluorobenzyl)amino)-1,3,5-triazin-2-yl)-N-(3-(pyrrolidin-1-yl)propyl)piperidine-3-carboxamide (002). A mixture of the previous compound (250 mg, 0.731 mmol), crude N-(3-(pyrrolidin-1-yl)propyl)piperidine-3-carboxamide (296 mg, 1.24 mmol), DIPEA (482 mg, 3.74 mmol) and 1,4-dioxane (10 mL) was heated at 90° C. for 24 h. The followed work up and isolation of the title product proceeded as described above (005), white amorphous solid, yield 120 mg (30%). ¹H NMR (400 MHz, DMSO-d₆) δ 7.79 (br. t, J=5.2 Hz, 1H), 7.35-7.25 (m, 2H), 7.13 (d, J=7.7 Hz, 1H), 7.09 (d, J=10.0 Hz, 1H), 7.01 (t, J=8.5 Hz, 1H), 4.75-4.52 (m, 4H), 4.39 (d, J=6.1 Hz, 2H), 3.60-3.40 (m, 8H), 3.06 (dd, J=12.5, 6.6 Hz, 2H), 2.74 (t, J=12.0 Hz, 2H), 2.44-2.25 (m, 7H), 1.70-1.60 (m, 6H), 1.58-1.50 (m, 2H), 1.39 (br. s, 2H). HRMS (ESI-TOF): [C₂₇H₄₁FN₈O₃+H]⁺. found 545.3378. calcd. 545.3358.

### Example Compound 006: 3-((4-(3-(4-benzylpiperidine-1-carbonyl)piperidin-1-yl)-6-((3-(4-fluorophenyl)propyl)amino)-1,3,5-triazin-2-yl)amino)propanoic Acid (006)

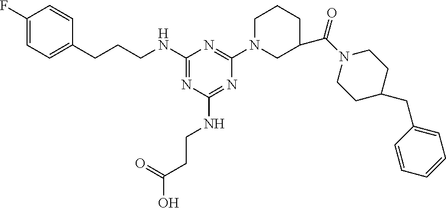 [SMILES: O=C(O)CCNc1nc(NCCCc2ccc(F)cc2)nc(N2CCCC(C(=O)N3CCC(Cc4ccccc4)CC3)C2)n1]

The necessary amine (4-benzylpiperidin-1-yl) (piperidin-3-yl)methanone was prepared using 2-nitrobenzenesulfonyl (2-Ns) strategy starting from (+)-ethyl nipecotate and 4-benzylpiperidine over 2-Ns-nipecotic acid and its acyl chloride followed by 2-Ns removal as described for compound (005) and used without chromatographic purification. ¹H NMR (400 MHz, CDCl₃) δ 7.37-7.06 (m, 5H), 4.59 (d, J=13.0 Hz, 1H), 3.91 (d, J=13.1 Hz, 1H), 3.73 (br. s, 2H), 3.18-2.40 (m, 8H), 1.93-1.47 (m, 7H), 1.21-1.05 (m, 2H), ˜90% purity.

4-Benzylpiperidin-1-yl) (1-(4-chloro-6-((3-(4-fluorophenyl)propyl)amino)-1,3,5-triazin-2-yl)piperidin-3-yl)methanone was prepared using the same one-pot procedure from cyanuric chloride (231 mg, 1.25 mmol), the crude previous amine (400 mg) and finally 3-(4-fluorophenyl) propan-1-amine (191 mg, 1.25 mmol) in the presence of DIPEA (348 mg, 2.70 mmol, total). Yield 250 mg (32%). ¹H NMR (400 MHz, CDCl₃) δ 7.34-7.08 (m, 7H), 6.98 (t, J=7.9 Hz, 2H), 5.67-4.93 (br. m, 1H), 4.88-4.53 (m, 3H), 4.11-3.84 (m, 1H), 3.50-3.25 (m, 2H), 3.10-2.80 (m, 3H), 2.70-2.45 (m, 6H), 1.99-1.70 (m, 9H), 1.20-1.12 (br. m., 2H). A mixture of rotamers.

Methyl 3-((4-(3-(4-benzylpiperidine-1-carbonyl)piperidin-1-yl)-6-((3-(4-fluorophenyl)-propyl)amino)-1,3,5-triazin-2-yl)amino)propanoate was prepared as described for compound 002 from the previous triazinyl chloride (240 mg, 0.436 mmol), β-alanine methyl ester hydrochloride (160 mg, 1.14 mmol) DIPEA (207 mg, 1.60 mmol). Yield 155 mg (58%), a white amorphous solid. ¹H NMR (400 MHz, CDCl₃) δ 7.34-7.10 (m, 7H), 7.01-6.94 (m, 2H), 5.14 (br. s, 1H), 4.92-4.58 (m, 4H), 4.08-3.96 (m, 1H), 3.71 and 3.69 (s, total 3H), 3.69-3.61 (m, 2H), 3.45-3.31 (m, 2H), 3.03-2.82 (m, 2H), 2.80-2.43 (m, 9H), 1.94-1.68 (m, 9H), 1.24-1.12 (m, 2H). A mixture of rotamers.

The title compound (006) was prepared by acidic hydrolysis of the previous methyl ester (140 mg, 0.226 mmol) as described above. Yield 90 mg (67%), a white amorphous solid. ¹H NMR (400 MHz, DMSO-d₆, 100° C.) δ 8.0-7.5 (br, 1H), 7.32-7.21 (m, 7H), 7.06 (t, J=8.7 Hz, 2H), 4.51 (t, J=11.2 Hz, 2H), 4.13 (s, 2H), 3.54 (t, J=6.4 Hz, 2H), 3.35 (br. t, J=5.6 Hz, 2H), 3.11-2.91 (m, 2H), 2.83-2.73 (br, 2H), 2.66 (t, J=7.5 Hz, 2H), 2.58-2.45 (m, 7H), 1.94-1.44 (m, 9H), 1.18-1.12 (m, 2H). HRMS (ESI-TOF): [C₃₃H₄₂FN₇O₃+H]⁺. found 604.3435. calcd. 604.3406.

### Example Compound 004: 1-(4-(bis(2-hydroxyethyl)amino)-6-((3-fluorobenzyl)amino)-1,3,5-triazin-2-yl)-N-(4-bromo-3-(trifluoromethyl)phenyl)piperidine-2-carboxamide (004)

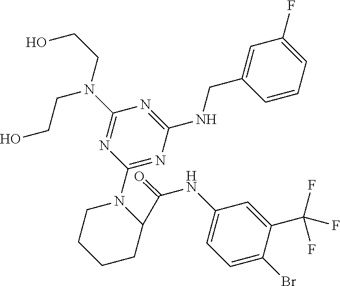 [SMILES: O=C(Nc1ccc(Br)c(C(F)(F)F)c1)C1CCCCN1c1nc(NCc2cccc(F)c2)nc(N(CCO)CCO)n1]

The necessary amine N-(4-bromo-3-(trifluoromethyl)phenyl)piperidine-2-carboxamide was prepared using 2-nitrobenzenesulfonyl strategy starting from (+)-ethyl piperidine-2-carboxylate and N-(4-bromo-3-(trifluoromethyl) aniline close related to compound 005. A cream amorphous solid. ¹H NMR (400 MHz, CDCl₃) δ 9.13 (br. s, 1H), 7.91 (d, J=2.5 Hz, 1H), 7.75 (dd, J=8.7, 2.4 Hz, 1H), 7.64 (d, J=8.7 Hz, 1H), 3.39 (dd, J=9.3, 3.6 Hz, 1H), 3.06 (dt, J=11.5, 3.6 Hz, 1H), 2.85-2.75 (m, 1H), 2.09-1.98 (m, 1H), 1.93-1.80 (m, 1H), 1.72 (br. s, 1H), 1.67-1.42 (m, 5H).

N-(4-Bromo-3-(trifluoromethyl)phenyl)-1-(4-chloro-6-((3-fluorobenzyl)amino)-1,3,5-triazin-2-yl)piperidine-2-carboxamide was prepared following the above mentioned one-pot procedure (002) from cyanuric chloride (185 mg, 1.00 mmol), N-(4-bromo-3-(trifluoromethyl)phenyl)piperidine-2-carboxamide (420 mg, 1.20 mmol) and then 3-fluorobenzylamine (139 mg, 1.10 mmol) in the presence of DIEPA (322 mg, 2.5 mmol total). Yield 320 mg (54%), a beige foam. ¹H NMR (400 MHz, CDCl₃) δ 8.7 and 8.1 (br. s, 1H), 7.95-7.70 (br. m, 1H), 7.66-7.58 (m, 2H), 7.48-7.29 (m, 1H), 7.25-6.24 (br. m, 4H), 5.62-5.32 (br. m, 1H), 4.82-4.49 (m, 3H), 3.02-2.87 (m, 1H), 2.45-2.30 (m, 1H), 2.00-1.43 (m, 5H). A mixture of rotamers.

The title compound (004) was prepared from the previous compound (280 mg, 0.478 mmol), diethanolamine (106 mg, 1.00 mmol) and DIEPA (140 mg, 1.09 mmol) in 1,4-dioxane (10 mL) as described for 005. Yield 102 mg (33%), a white amorphous solid. ¹H NMR (400 MHz, DMSO-d₆) δ 10.3-10.2 (br. m, 1H), 8.20 and 8.05 (br. s, 1H), 7.91-7.56 (m, 2H), 7.48-6.65 (m, 5H), 5.32 (d, J=61.8 Hz, 1H), 4.75-4.38 (br. m, 4H), 3.72-3.32 (m, 10H), 3.30-3.02 (br. m, 1H), 1.80-1.55 (m, 3H), 1.45-1.29 (m, 2H). A mixture of rotamers. HRMS (ESI-TOF): [C₂₇H₃₀F₃N₇O₃+H]⁺. found 658.1625. calcd. 658.1582.

### Example Compound 003: (1-(4-(bis(2-hydroxyethyl)amino)-6-((3-fluorobenzyl)amino)-1,3,5-triazin-2-yl)piperidin-2-yl)(morpholino)methanone (003)

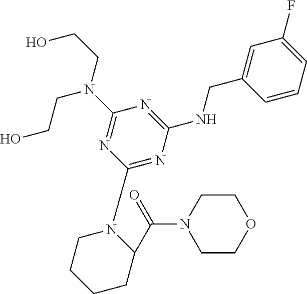 [SMILES: O=C(C1CCCCN1c1nc(NCc2cccc(F)c2)nc(N(CCO)CCO)n1)N1CCOCC1]

The necessary amine morpholino (piperidin-2-yl)methanone was prepared from (±)-N-Boc-piperidine-2-carboxylic acid and morpholine using standard mixed anhydride protocol (IBCF, NMM, DCM; column chromatography).

(1-(4-chloro-6-((3-fluorobenzyl)amino)-1,3,5-triazin-2-yl)piperidin-2-yl)(morpholino)methanone was prepared following the above mentioned one-pot procedure (002) from cyanuric chloride (185 mg, 1.00 mmol), the previous amine (220 mg, 1.11 mmol) and then 3-fluorobenzylamine (139 mg, 1.10 mmol) in the presence of DIPEA (322 mg, 2.5 mmol total). Yield 165 mg (38%), a white foam. ¹H NMR (400 MHz, DMSO-d₆) δ 8.5-8.1 (four t, 1H), 7.43-7.32 (m, 1H), 7.20-6.99 (m, 3H), 5.45-5.35 (m, 1H), 4.77-4.22 (m, 3H), 3.74-3.07 (m, 14H), 1.96-1.28 (m, 6H). A mixture of four rotamers.

The title compound (003) was prepared from the previous compound (155 mg, 0.357 mmol), diethanolamine (106 mg, 1.00 mmol) and DIEPA (140 mg, 1.09 mmol) in 1,4-dioxane (10 mL) as described for 005. Yield 108 mg (60%), a white amorphous solid. ¹H NMR (400 MHz, DMSO-d₆) δ 7.37-7.28 (m, 1H), 7.19-6.97 (m, 4H), 5.43 (br. s, 1H), 4.78-4.20 (m, 5H), 3.74-3.06 (m, 17H), 1.94-1.21 (m, 6H). A mixture of rotamers. HRMS (ESI-TOF): [C₂₄H₃₄FN₇O₄+H]⁺. found 504.2747. calcd. 504.2729.

### Example Compound 001: (R)-1-(4-(bis(2-hydroxyethyl)amino)-6-((3-fluorobenzyl)amino)-1,3,5-triazin-2-yl)-N,N-diethylpyrrolidine-2-carboxamide (001)

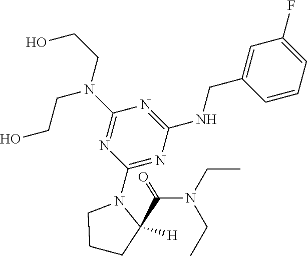 [SMILES: CCN(CC)C(=O)[C@H]1CCCN1c1nc(NCc2cccc(F)c2)nc(N(CCO)CCO)n1]

The necessary amine L-Pro-NEt₂ was prepared from Boc-L-Pro-OH and diethylamine using standard mixed anhydride protocol (IBCF, NMM, DCM; crystallization from Et₂O/hexane).

The title compound (001) was prepared from N-(4-chloro-6-(3-fluorobenzylamino)-1,3,5-triazin-2-yl) diethanolamine (as above for detailed compound 002, 171 mg, 0.50 mmol) and the previous amine (170 mg, 1.00 mmol) as described in that case. Yield 115 mg (48%), a white amorphous solid. ¹H NMR (400 MHz, DMSO-d₆, 100° C.) δ 7.32 (td, J=7.9, 6.1 Hz, 1H), 7.18-7.02 (br. m, 2H), 6.98 (td, J=8.4, 2.2 Hz, 1H), 7.0-6.4 (br., 1H), 4.80 (dd, J=8.6, 3.4 Hz, 1H), 4.46 (d, J=6.4 Hz, 2H), 4.33 (br. s, 2H), 3.72-3.14 (m, 14H), 2.24 (dq, J=12.1, 8.2 Hz, 1H), 2.03-1.71 (m, 3H), 1.29-0.96 (br. s, 6H). HRMS (ESI-TOF): [C₂₃H₃₄FN₇O₃+H]⁺. found 476.2796. calcd. 476.2780.

### Example 2: Biological Assays

### Materials and Methods

### Luciferase Reporter Assay

The HEK293E1a cells were seeded at density 1.2×10⁴ per well on 48-well plate in DMEM medium containing 10% fetal calf serum. After 24 hours cells were transfected with plasmid mix (300 ng/well) containing specified amounts of the p6×PORE-luc reporter, pEV-OBF1 and pCG-Oct1 effector, pBluescript II KS+ carrier, and pCMV-βgal (for standardization) as previously described. The transfections were carried out in three biological replicates, using Polyethylenimine (PEI 25K, Polysciences Inc.) at the ratio 2:1 to DNA. After 48 hours, cells were washed with PBS, lysed, and measured for luciferase activity, using the Bright-Glo luciferase assay system (Promega), and for β-galactosidase activity in a standard assay.

### Protein Expression and Purification

DNA sequences coding the POU domain of OCT1 (POU1) and BOB1 were PCR amplified using human cDNA as a template. Following primers were used for the amplification:

(SEQ ID NO: 4)
5′-TTAGGATCCGAGGAGCCCAGTGACCTTGA-3′
-POU1 forward,
(SEQ ID NO: 5)
5′-GCAAAGCTTAGTTGATTCTTTTTTCTTTCTGGCGG-3′
-POU1 reverse;
(SEQ ID NO: 6)
5′-TTAGGATCCATGCTCTGGCAAAAACCCAC-3′
-BOB1 forward,
(SEQ ID NO: 7)
5′-GCAAAGCTTCTAAAAGCCTTCCACAGAGAGAGT-3′
-BOB1 reverse.

The amplified fragments were cleaved with BamHI and HindIII and ligated into a pET28 vector. The resulting constructs contained in-frame N-terminal histidine tag. Protein expression was induced with 1 mM IPTG in E. Coli BL21 cells for 4 hours. Cells were harvested by centrifugation and lysed in buffer A1 (8 M urea, 0.1 M sodium phosphate buffer, 0.01 M Tris-Cl, pH 8.0, 5 mM β-mercaptoethanol). Cell lysates were applied to an Ni-NTA His·Bind Resin (Novagen), washed with buffer A2 (8 M urea, 0.1 M sodium phosphate buffer, 0.01 M Tris-Cl pH 6.3, 5 mM β-mercaptoethanol), and eluted with buffer A3 (8 M urea, 0.1 M sodium phosphate buffer, 0.01 M Tris-Cl pH 4.5, 5 mM β-mercaptoethanol). The protein samples were subjected to four-step dialysis against buffers with a two-fold decrease of urea concentration and a gradual increase of pH at each step. Final dialysis step was performed against PBS containing 5 mM β-mercaptoethanol for 12 hours. The POU1 domain was additionally purified with a Hi-Trap heparin-Sepharose column. The column was pre-equilibrated with buffer B (20 mM HEPES pH 7.6, 100 mM NaCl, 1 mM EDTA), the sample was loaded onto the column and then eluted with a linear gradient of NaCl from 100 mM to 500 mM in the same buffer. BOB1 was further purified on a Superdex 75 column pre-equilibrated with PBS containing 5 mM β-mercaptoethanol.

### Electrophoretic Mobility Shift Assay (EMSA)

Biotinylated PORE-containing dsDNA (underlined) probe was prepared by annealing oligonucleotides: 5′-biotin-AAGTTAAAATCACATTTGAAATGCAAATGGAAAAGCAAGGCCCG-3′ (SEQ ID NO: 8), and 5′-CGGGCCTTGCTTTTCCATTTGCATTTCAAATGTGATTTTAACTT-3′ (SEQ ID NO: 9) (Evrogen, Moscow) in 10 mM Tris HCl pH 8.0, 100 mM NaCl, 10 mM MgCl2 buffer by heating the solution to 95° C. for 5 min and then cooling it down slowly to room temperature in a water bath. Biotinylated PORED dsDNA probe (20 ng) was mixed with the purified protein samples (500 ng BOB1, 400 ng OCT1) in 10 μL of binding buffer containing Tris-HCl pH 7.6, 10% glycerol, 0.14 M NaCl, 0.5 mM EDTA, 0.1 mg/mL bovine serum albumin (BSA), 0.1 mg/mL poly(dI-dC) (Amersham), 5 mM DDT, and 1× protease inhibitor cocktail (Complete, Roche), then incubated for 20 min at room temperature. The samples were separated by electrophoresis in 6% polyacrylamide gel in 0.5×TBE buffer and transferred onto the nitrocellulose membrane. The membrane was blocked with 2.5% BSA in PBS for 30 min, incubated for 1 hour at room temperature with streptavidin-HRP (Sigma) diluted 1:10000 in blocking buffer, washed with blocking buffer for 30 min and twice with PBS-0.1% Tween-20 for 30 min. Chemiluminescence was visualised with SuperSignal West Pico Chemiluminescent Substrate (Thermo Scientific).

### Thermal Shift Assay (TSA)

POU1 and BOB1 proteins (each at a final concentration 5 μM), compound 005 (final concentration 10 μM), and 1× ProteOrange fluorescent dye (Lumiprobe, Russia) were mixed in triplicates in 100 μL of 0.1 M phosphate buffered saline pH 7.4 per and pipetted into 96-well 0.2-mL thin-wall PCR plates (Roche Life Science, Switzerland) sealed with optical-grade sealing film. Fluorescence data were acquired on a LightCycler® 96 Real Time PCR Instrument (Roche Life Science, Switzerland) with an excitation range of 470-533 nm. The fluorescence emission signal at 533-5572 nm was measured for data analysis. The temperature was held for 30 sec per degree from 37 to 98° C. (ramp rate of ˜0.5° C./min). The derivatives of the fluorescence signal as function of the temperature were used for further analysis.

### Kd Measurement

The study of the binding of compound 005 to POU1 protein was performed using a Varioskan™ LUX multimode microplate reader (Thermo Fisher Scientific, USA). The emission spectra were recorded in the wavelength range λ=310-450 nm at an excitation wavelength of 280 nm at a temperature of 298.15 K with an accuracy of ±0.3 K. The POU1 concentration was 3 μM, while the compound 005 concentration was within the range 3-39 μM with a 6-μM step. The calculations were carried out based on three parallel measurements.

### Primary B Cell Purification and Enrichment

Blood samples were obtained from healthy volunteers provided by Sanquin Blood Supply Foundation (Amsterdam, the Netherlands) or from Etablissement Français du Sang (Nantes, France) after written consent was obtained. Peripheral blood mononuclear cells (PBMCs) were isolated through Ficoll-Paque gradient centrifugation on Lymphoprep (Nycomed). Total B cells were isolated from freshly purified PBMCs by negative selection (B cell Isolation Kit II; Miltenyi Biotech, 130-091-151) on magnetic columns using autoMACS Pro Separator according to the manufacturer's instructions. Subsequently, memory and naïve B cells were isolated from negatively-selected CD19⁺ B cells by positive and negative selection with CD27 MicroBeads (Miltenyi Biotech, 130-051-601) using autoMACS Pro Separator according to the manufacturer's instructions. Ultimately, purity of the MACS-isolated CD19⁺ B cells, CD19⁺CD27⁺IgD⁻ memory B cells and CD19⁺CD27⁻IgD⁺ memory B cells was assessed by flow cytometry; purity was >90% for B cells, >65% for memory B cells, and >85% for naive B cells. All cell cultures were carried out in RPMI 1640 medium (Thermo Fisher Scientific) supplemented with 10% FCS, L-glutamine, and penicillin/streptomycin. Cells were incubated at 37° C. in a 5% CO₂ atmosphere.

### Cell Proliferation Dye (CPD) Labelling

Cells were resuspended in PBS and labelled with Cell Proliferation Dye eFluor 450 (CPD) (eBioscience) at a final concentration of 10 μM at 37° C. for 10 min, kept on ice for 5 min, and washed twice with 10 mL of ice-cold RPMI 1640 10% FCS medium.

### In Vitro Plasmablast Differentiation and Ig Production

B cells were stimulated to differentiate into plasmablasts using a two-phase cell culture protocol as previously described. Briefly, B cells were seeded at 4×10⁵ cells/mL in 24-well plates and activated during 4 days with 50 ng/mL recombinant human soluble CD40L (R&D Systems), 2.5 μg/mL CpG oligodeoxynucleotide (ODN) 2006 (Amidogen or Cayla Invivogen), 2 μg/mL F(ab′)₂ fragment goat anti-human IgA+IgG+IgM (H+L) polyclonal Abs (anti-BCR) (Jackson ImmunoResearch Laboratories), and 50 U/mL human IL-2 (SARL Pharmaxie or Novartis). Day 4-activated B cells were washed and cultured at the same concentration for three additional days in a fresh medium containing 50 U/mL human IL-2, 10 ng/mL human IL-4 (R&D Systems), and 10 ng/mL human IL-10 (R&D Systems) to induce B cell differentiation into plasmablasts. At days 0, 4 and 7, B cells were stained with viability marker LIVE/DEAD Fixable Aqua Dead Cell Stain (Thermofisher) to identify dead cells, followed by extracellular staining with anti-human mAbs: anti-CD20 FITC, anti-IgD BUV395, anti-CD27 BUV737, anti-CD38 BV605 (all from BD Biosciences) and analyzed by flow cytometry (FACSCelesta™, BD Biosciences). In some experiments, B cell apoptosis was evaluated by intracellular active caspase-3 staining (Caspase-3-PE, BD Biosciences).

Immunoglobulin isotypes were quantified using the Human Immunoglobulin Isotyping LEGENDplex 8-plex kit (BioLegend) according to the manufacturer's instructions. Data were collected on the BD FACSCanto™ II flow cytometer and analyzed using LEGENDplex Data Analysis Software.

High-Throughput Multiplex qPCR

mRNA was extracted from freshly thawed cell lysates using the RNEasy micro kit (Qiagen) according to the manufacturer's instructions. cDNA was synthesised using oligo-dT and Omniscript RT (Qiagen) according to the manufacturer's instructions. Gene expression levels of 46 B-cell associated genes were evaluated by qRT-PCR using the Biomark™ HD System and the 46.48 GE Dynamic Arrays (Fluidigm) following the manufacturer's protocol and following gene expression assays (all from ThermoFisher): AICDA (Hs00221068_m1), B2M (Hs99999907_m1), BACH2 (Hs00222364_m1), BCL2L1 (Hs00236329_m1), BCL6 (Hs00153368_m1), CCL3 (Hs00234142_m1), CCND1 (Hs00765553_m1), CCND2 (Hs00153380_m1), CD27 (Hs00386811_m1), CD38 (Hs01120071_m1), CD40 (Hs00374176_m1), CD79a (Hs00233566_m1), CD80 (Hs01045163_m1), CD86 (Hs99999104_m1), Ciita (Hs00172106_m1), CXCR4 (Hs00607978_s1), ERN1 (Hs00176385_m1), ETS1 (Hs00428293_m1), EZH2 (Hs00172783_m1), HPRT1 (Hs02800695_m1), ID2 (Hs04187239_m1), ID3 (Hs00171409_m1), IgHG1 (Hs00378340_m1), IgHG3 (Hs00941519_g1), IGHM (Hs00378435_m1), IL2RA (Hs00907779_m1), IL6 (Hs00174131_m1), IRF4 (Hs00180031_m1), IRF8 (Hs01128710_m1), MEF2C (Hs00231149_m1), MKI67 (Hs01032443_m1), MS4A1 (Hs00544819_m1), MYC (Hs00153408_m1), PAX5 (Hs00277134_m1), POU2AF1 (Hs01573371_m1), POU2F2 (Hs00922172_m1), PRDM1 (Hs00153357_m1), RELA (Hs00153294_m1), RELB (Hs00232399_m1), SDC1 (Hs00896423_m1), SLAMF1 (Hs00234149_m1), SPI1 (Hs02786711_m1), SPIB (Hs00162150_m1), STAT3 (Hs01047580_m1), SYK (Hs00895377_m1), TLR4 (Hs00370853_m1), TLR9 (Hs00152973_m1), XBP1s (Hs03929085_g1). Data were normalised using the geometric average of two housekeeping genes (HPRT1, B2M) and the delta/delta Ct method. Unsupervised hierarchical gene clustering based on Euclidian distance and complete-linkage method was generated in R (v4.2.2) using pheatmap (v1.0.12) package.

### GZMB⁺ B Cell Expansion and T Cell Suppression Assay

Primary total human B cells (1×10⁶ cells/mL) were prestimulated for ˜24 h with the cocktail containing 10 ng/mL of IL-21 (Miltenyi Biotec), 5 μg/mL of F(ab′)₂ anti-BCR Abs (Jackson ImmunoResearch Laboratories Inc.), 50 ng/ml of CD40L (R&D systems), 1 μg/mL of CpG oligonucleotides (ODN 2006, Invivogen), and 50 U/mL of IL-2 (Novartis) as previously described, in the presence of vehicle (DMSO) or 10 μM compound 005. On day 3, cells were stained with Fixable Viability Dye (FVD) eFluor 450 to identify dead cells, followed by staining for CD19 and intracellular granzyme B (GZMB) to evaluate B cell GZMB expression. The capacity of B cells to suppress effector T cell proliferation was assessed as previously described. For autologous T cell isolation, PBMCs from the same donor were kept overnight in culture medium at 4° C. The next day, CD4+ T cells were isolated by negative selections (human CD4+ T cell isolation kit, Miltenyi Biotech, 130-096-533) on magnetic columns using autoMACS Pro Separator according to the manufacturer's instructions. Subsequently, CD25⁺ T cells were depleted from the CD4⁺ T cells using CD25 microbeads (Miltenyi Biotech, 130-092-983) using autoMACS Pro Separator to obtain CD4⁺CD25⁻ effector T cells. B cells were then washed, resuspended in a fresh culture medium and co-cultured with autologous CPD-labelled CD4⁺CD25⁻ effector T cells for three days in the presence of CD3/CD28 Dynabeads (Invitrogen) at a T cell/bead ratio of 1:1 and different concentrations of compound 005. A total of 1×10⁵ B cells were co-cultured with 0.5×10⁵ autologous T cells in a total volume of 150 μL/well in a 96-well, U-bottom plate. T cell proliferation was evaluated as the percentage of CPD-low T cells on day 3.

### ScRNA Sequencing

B cells from 2 healthy volunteers were sorted with Human B Cell Isolation Kit II. Separation was performed on an AutoMACS pro Separator following supplier instructions. GZMB⁺ Bregs were induced with 50U/mL rhIL-2, (Proleukine—Novartis); 10 ng/mL rhIL-21, R&D systems; 5 μg/mL goat anti human IgG+A+M (H+L) F(ab)′2 (Jackson ImmunoResearch); 1 μg/mL CpG oligodeoxynucleotides ODN 2006—Invivogen); 50 ng/mL recombinant human soluble CD40L (R&D systems) in complete medium for 24h in an incubator at 37° C. 5% CO₂ in 6-well plate, 5·10⁶ cells/5 mL/well. At the end of the cultures, cells were marked with viability dye (Fixable Viability Dye eFluor 450, 1/1000 in PBS without azide or protein, Invitrogen) for 25 min. Living cells were sorted on an ARIA III (BD Biosciences). Living cells were marked with anti-β2-microglobulin or anti-CD-293 conjugated DNA sequences (HashTag Oligonucleotide, HTO) by following CITE-seq protocols (https://www.biorxiv.org/content/10.1101/237693v1). Cells were then pooled at equivalent concentrations and 20,000 total cells were sequenced on the Nova-Seq 6000 (Illumina) at the GenoBird plateform (IRS-UN, CHU Nantes). Data were analyzed with R, Rstudio, Seurat v 4.0.2 and Graphpad Prism.

### T Cell Recall Assay

Total PBMCs were plated into 96-well round-bottom plates at 0.5×10⁶ cells/well and were stimulated with anti-CD3/CD28 Dynabeads (Gibco Life Technologies) at a bead/cell ratio of 0.2:1 in the presence of compound 005 (10 μM) or vehicle (DMSO) for 6 hours to induce polyclonal primary responses. To induce recall responses, cells were stimulated with beads for two days, allowed to rest for eight days in medium supplemented with IL-2 (10 U/mL) and re-stimulated with anti-CD3/CD28 beads for the last 6 hours in the presence of compound 005 (10 mM) or vehicle. Expression of Th1-, Th2-, Th17-, and Treg-associated cytokines and transcription factors was assessed by qPCR. For intracellular staining, cells were re-stimulated for 10 hours with anti-CD3/CD28 beads, and Brefeldin A (10 μg/mL, Sigma) was added to the medium 4 hours after the start of re-stimulation. Dead cells were excluded using the LIVE/DEAD® Fixable Aqua Dead Cell Stain Kit (Life Technologies). Subsequently, cells were surface-stained with CD3-BUV395, CD4-BUV737, CD8-Violet650 (all three from BDHorizon), and CD161-PE-Cy7 (from Biolegend) at 4° C. for 30 min, followed by incubation with Fixation/Permeabilization working solution (eBioscience), washed with Permeabilization Buffer and stained with IL-17A-PE and IFNγ-BV711 (both from BD Biosciences). Cells were resuspended in ice-cold FACS buffer (0.5% BSA, 2 mM EDTA and 50 U/mL penicillin/streptomycin in PBS). Flow cytometry data were collected on BD FACSCelesta™ and analyzed using FlowJo™ cell analysis software (FlowJo, LLC).

qRT-PCR

Total RNA was extracted from PBMCs using the RNeasy Micro Kit (Qiagen) according to the manufacturer's protocol. RNA was processed for cDNA synthesis (Fermentas) and analyzed by qPCR with TaqMan gene expression assays for IFNG (Hs00989291_m1), IL-2 (Hs00174114_m1), TNF (Hs00174128_m1), TBX21 (Hs00203436_m1), IL-4 (Hs00174122_m1), GATA3 (Hs00231122_m1), IL-17A (Hs00174383_m1), IL-17F (Hs00369400_m1), IL-21 (Hs00222327_m1), IL-22 (Hs01574154_m1), RORC (Hs01076122_m1), IL-10 (Hs00961622_m1), FOXP3 (Hs01085834_m1), POU2AF1 (Hs01573371_m1) (Thermofisher) using Real-Time PCR QuantStudio3 PCR System (Applied Biosystems). Expressions of all genes were normalized to the expression of GAPDH (4310884E) as housekeeping gene and presented as fold change relative to the reference sample.

### Statistical Analysis

Differences between groups were analyzed using a one-way ANOVA with Greenhouse-Geisser correction, followed by multiple comparisons with Bonferroni correction was performed (p<0.05). Paired ratio test was performed for in vitro PBMCs stimulation assay (p<0.05). Values are expressed as mean and SD or median and IQR range, according to criteria for (non) parametric analysis. Prism version 7 software (GraphPad Software) was used for statistical testing.

### Results

### Compound 005 Antagonizes BOB1/OCT1-Dependent Transcriptional Activation

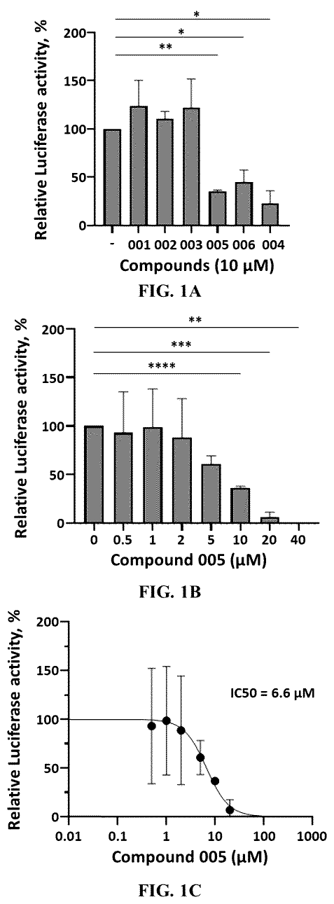

BOB1 has been described as a coactivator acting in cooperation with the POU-domain proteins OCT1 and OCT2 bound to distinct DNA-motifs either in monomeric or dimeric configurations. One class of these motifs (called Palindromic Oct-Recognition Elements, or PORE) has been shown to serve as a platform for OCT1 dimer assembly in a specific dimeric configuration that allows BOB1 recruitment, providing thereby transcriptional activation. As an initial functional assay, we examined the ability of the compounds according to the present invention to affect BOB1-OCT1-PORE-mediated transcriptional activation in transient transfection luciferase assay. To this end, we made use of previously generated 6×PORE-luc and reporter plasmid, in which hexamers of the PORE were cloned upstream of the thymidine kinase (tk) minimal promoter and luciferase gene. In preliminary tests we found that transfection of HEK293 cells with BOB1-expressing plasmid alone could drive the luc-reporter transcription in a dose-dependent manner (data not shown). Furthermore, the addition of a plasmid expressing exogenous OCT1 exerted an inhibitory effect on both basal and BOB1-mediated transcription (data not shown). This observation is likely due to the fact that HEK293 cells, like all other mammalian cell types, express OCT1, and further addition of exogenous OCT1 in these cells shifts the BOB1/OCT1 equilibrium out of optimal range, resulting in squelching effect, and in our experimental setup, in about 3-fold stimulation, as compared to that by endogenous OCT1 alone (FIG. 1A). Out of six synthesized compounds, compound 005, compound 006, and compound 004 at the concentration of 10 μM in the culture medium significantly attenuated BOB1/OCT1-driven transcription to the level mediated by endogenous OCT1, whereas the other compounds showed no inhibitory activity at this concentration (FIG. 1A). Compound 005 was selected for further detailed investigation. We have certified that compound 005 suppresses transcriptional activation in a dose-dependent manner within the concentration range from 0.5 to 40 μM (FIG. 1B-C). The inhibitory effect of compound 005 was readily observed in MTT assay starting at the concentration 10 μM and above, while remained non-toxic or mildly toxic for HEK293 cells at concentrations up to 40 μM, while for NIH3T3 cells and mouse embryonic fibroblasts (MEFs) compound 005 showed toxicity only at 80 μM and above (data not shown).

### Compound 005 Binds OCT1 In Vitro

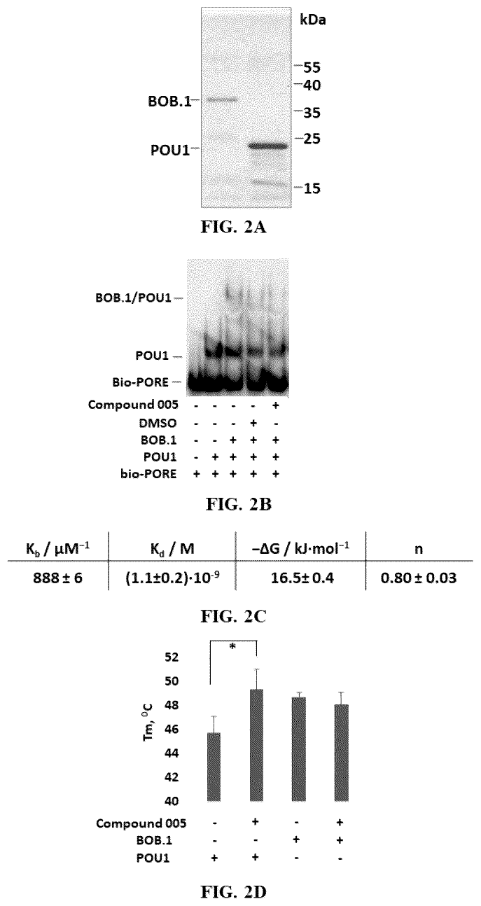

To assess whether compound 005 binds OCT1 in vitro, we have bacterially expressed and purified 6×His-tagged full-length BOB1 protein and the POU domain of OCT1 (POU1), which has been previously shown to mediate this interaction. The two tagged proteins of predicted molecular weight 31 kDa for BOB1 and 22 kDa for POU1 were expressed, purified to near homogeneity using Ni-NTA resin and refolded (FIG. 2A). Subsequently, their ability to form the ternary complex was verified by electrophoretic mobility shift assay (EMSA), using biotin-labelled PORE-containing dsDNA as a probe. POU1 forms on this sequence monomer and dimer complexes with the clear shift toward the latter (FIG. 2B, lane 2). In the presence of BOB1, a ternary complex is formed (FIG. 2B, lane 3). The preliminary test suggests that the bacterially produced POU1 and BOB1 are properly refolded and have acquired at least some of their biochemical activities. Unexpectedly, however, the presence of compound 005 in the binding reaction, even at a concentration (100 μM), has not affected the ternary BOB1/POU1/PORE complex (FIG. 2B, lane 5), indicating that either the EMSA conditions are not permissive for compound 005 binding or compound 005 mode of action does not involve a disruption of the ternary complex—these possibilities will be discussed below.

To determine the binding constant (Kb) and the number of binding sites (n) within compound 005 complex with POU1, dependences were plotted in Hill's coordinates:

$$1 g \frac{{F}_{0} - F}{F} = 1 g {K}_{b} + n 1 gQ$$

The obtained Kd value of 10⁻⁹, typical for drug-target interaction, confirms that compound 005 is able to form a complex with POU1 in solution. Gibb's energies changes were calculated based on the Kb value. The obtained negative value of ΔG indicates that this binding is thermodynamically favorable (FIG. 2C). Next, thermal shift assay (TSA) revealed that the addition of compound 005 increased POU1 melting temperature by about 4° C. (FIG. 2D).

In sum, the obtained results provide a compelling argument that compound 005 binds OCT1 in vitro with an affinity typical for drug-target interactions. This set of data also provides a solid biochemical rationale for further investigation of compound 005 biological effects in vivo.

### Compound 005 Suppresses T-Dependent B Cell Terminal Differentiation in a Dose-Dependent Manner

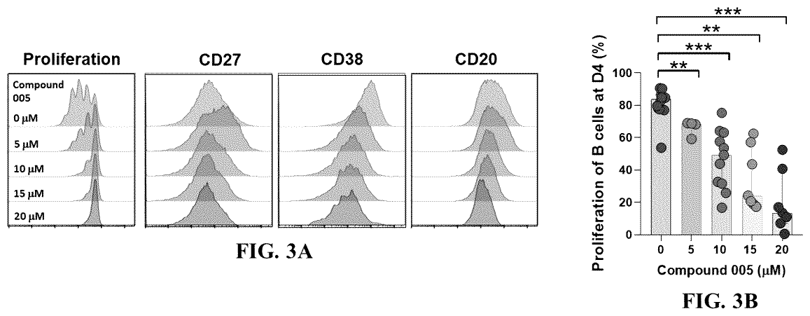

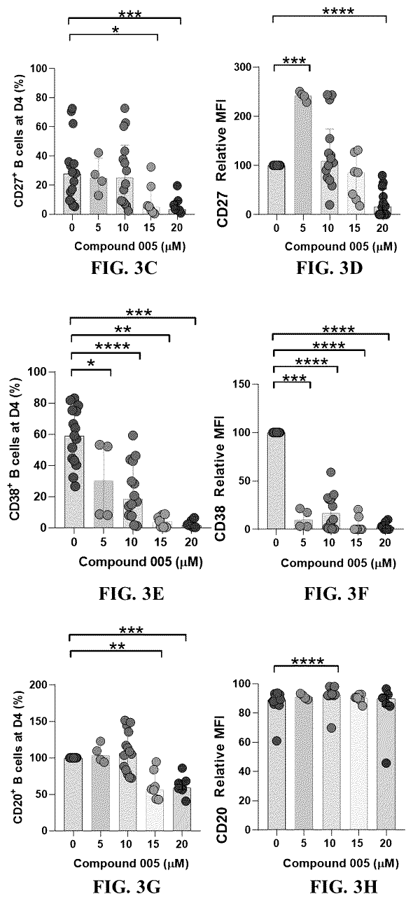

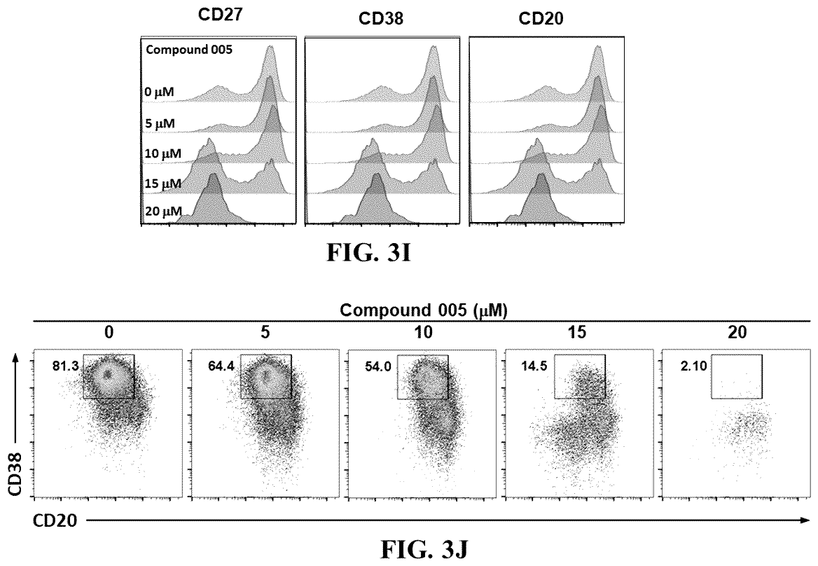

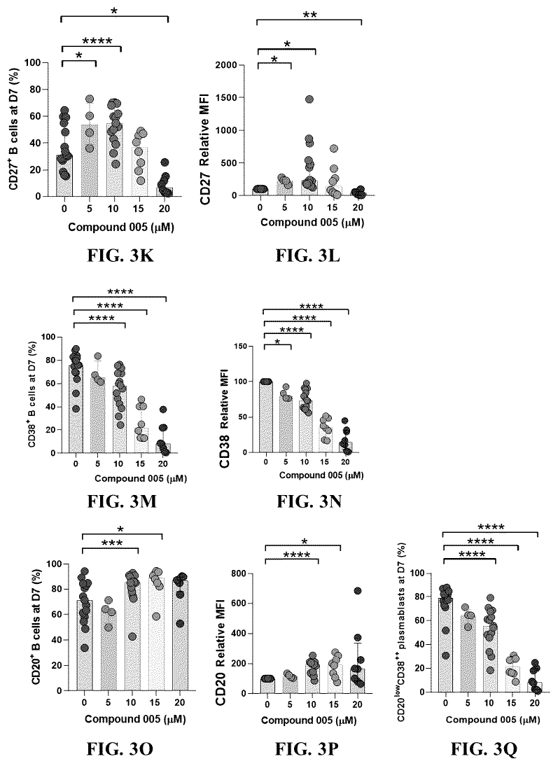

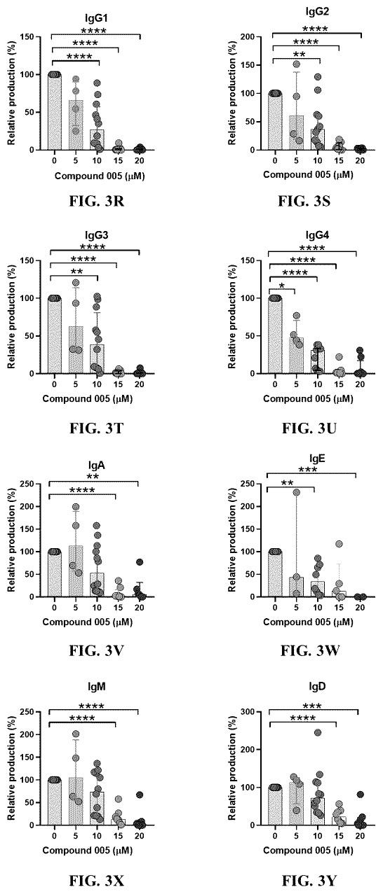

Previous studies demonstrated that the absence of functional BOB1 during the T-cell dependent response causes tremendous functional consequences such as a lack of germinal center formation, block of plasma cell differentiation and a severely reduced production of isotype switched antibodies to T-cell dependent antigens in vitro and in vivo. Therefore, we set out to investigate whether and how interference with BOB1 function by compound 005 would affect T-cell-dependent B cell differentiation employing a previously established in vitro model. This two-step human B cell differentiation model combines BCR signal, TLR activation, T cell help, and cytokines to mimic the molecular dynamic of in vivo germinal center B cell maturation. During the activation phase (days 0-4), B cells are first cultured with F(ab′)₂ anti-Ig(A+G+M), CD40L, CpG, and IL-2. On day 4, activated B cells are washed and then reseeded with IL-2, IL-4, and IL-10 in the absence of CD40L. As CD27+ memory B cells possess the intrinsic ability to undergo rapid terminal differentiation toward plasma cells in contrast to CD27 naïve B cells, we focused on this cell population. We analyzed the effect of BOB1 inhibition by compound 005 on B proliferative capacities at day 4 and the ability to differentiate to CD20^{lo}CD38^{hi} plasmablasts at day 7. Flow cytometric data analysis revealed that during the activation phase, a proliferation burst was distinguished with the majority of the cells cultured in the control condition (DMSO) (mean±SD of 80.9±9.7%) as assessed by the dilution of the cell proliferation dye at day 4 (FIG. 3A). Control B cells acquired expression of activation and maturation marker CD38 (data not shown), leading to the emergence of CD38^{hi} precursors that differentiate towards CD20^{lo}CD38^{hi} plasmablasts in the second phase of the model (mean±SD of 74.8±14.9%) (FIG. 3I). The addition of compound 005 to cell culture resulted in significant suppression of B cell-proliferative capacity in a dose-dependent fashion as compared to control B cells (66.3±4.8% (P=0.0063), 46.7±18.3% (P=0.0003), 34.9±19.2% (P=0.002), 20.4±19.0% (P=0.0002) for 5, 10, 15 and 20 μM of compound 005, respectively) (FIG. 3B). We also observed a similar dose-dependent suppressive effect of compound 005 on the expression of CD38 marker that led to the reduction in the expression mean and frequencies of CD38⁺ B cells during both activation and differentiation phases (mean±SD of 58.4±18.4% in control versus 30.5±25.6% (P=0.069), 23.9±18.3 (P=0.0001), 4.3±3.7% (P=0.0019), and 2.5±2.2 (P=0.0003) for 5, 10, 15 and 20 μM of compound 005, respectively, at day 4) (FIG. 3E-F) and (73.6±13.9% in control versus 68.9±10.2% (P=0.7224), 55.5±16.1 (P<0.0001), 26.3±13.9% (P<0.0001), and 13.0±12.9 (P<0.0001) for 5, 10, 15 and 20 μM of compound 005, respectively, at day 7) (FIG. 3M-N). In contrast, cells cultured in the presence of 10 μM (81.7±12.4% (P=0.001)), and 15 μM (85.4±11.7% (P=0.02)) of compound 005 exhibited elevated expression of CD20 marker compared to the control cells (69.8±16.8%) (FIG. 30-P). Consequently, inhibition of BOB1 induced a significant dose-dependent reduction in CD20^{lo}CD38^{hi} plasmablasts generation (median percentage decrease 30.3% (P<0.0001), 70.5% (P<0.0001), and 85.2% (P<0.0001) for 10, 15 and 20 μM of compound 005, respectively at day 7 (FIG. 3Q).

The effect of compound 005 on CD38 was rather specific, as we did not observe a similar decrease in CD27-positive cells in the cultures. In particular, at day 4, only high concentrations of compound 005 significantly decreased frequencies of CD27⁺ cells (29.9±22.5% in control versus 9.5±11.7% (P=0.015) and 6.4±6.4 (P=0.0009) for 15 and 20 μM of compound 005, respectively) (FIG. 3C-D). However, on day 7, the relative proportion of CD27⁺ cells was increased in the presence of 5 μM and 10 μM of compound 005, but reduced in cultures with 20 μM of the inhibitor (36.9±16.5% in control versus 54.3±15.9% for 5 μM (P=0.037) 53.6±14.2% (P<0.0001) for 10 μM of compound 005 and versus 9.4±8.1% for 20 μM compound 005 (P=0.01) (FIG. 3K-L).

To further validate the suppression of plasma cell differentiation upon BOB1 inhibition, we next assessed whether the dose-dependent decrease in the frequency of CD20^{lo}CD38^{hi} plasmablasts in response to the inhibition of BOB1-OCT(s) interaction by compound 005 correlates with the reduction in the secretion of antibodies. The multiplex immunoassay revealed that the titers of all immunoglobulin subclasses were strongly suppressed in the presence of 15 and 20 u M of compound 005 (FIGS. 3R-Y). However, 10 μM of compound 005 had a distinct effect on the immunoglobulin classes as a significant decrease in IgG1 (FIG. 3R), IgG2 (FIG. 3S), IgG3 (FIG. 3T), and IgG4 (FIG. 3U) but not IgA (FIG. 3V), IgE (FIG. 3W), IgM (FIG. 3X), and IgD (FIG. 3Y) production was observed.

We repeated similar experiments with CD27 B cells. The percentage of CD20^{lo}CD38^{hi} cells was significantly lower in this case (mean±SD of 21.5±15.6% in naïve B cells versus 72.2±15.7% in memory, P<0.0001) in line with the concept that naïve B cells are less amenable to plasma cell differentiation. Yet, obtained results revealed a comparable dose-dependent effect of compound 005 on proliferation, CD38 and CD20 expression and plasmablast differentiation (data not shown).

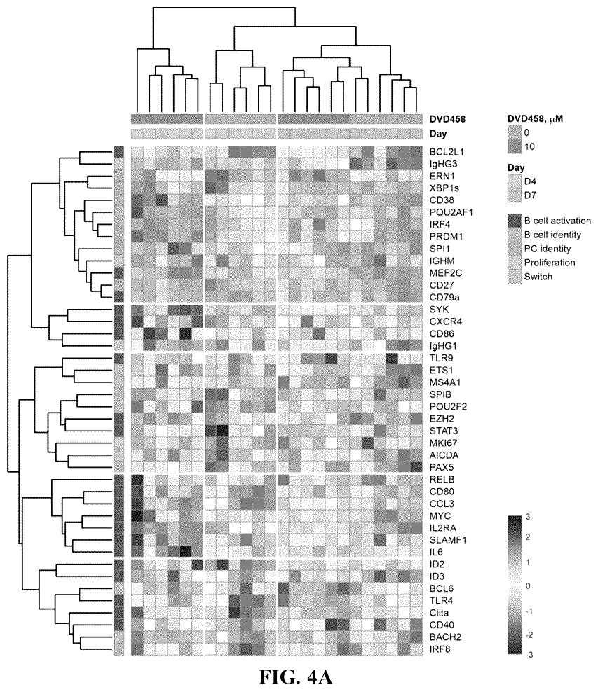

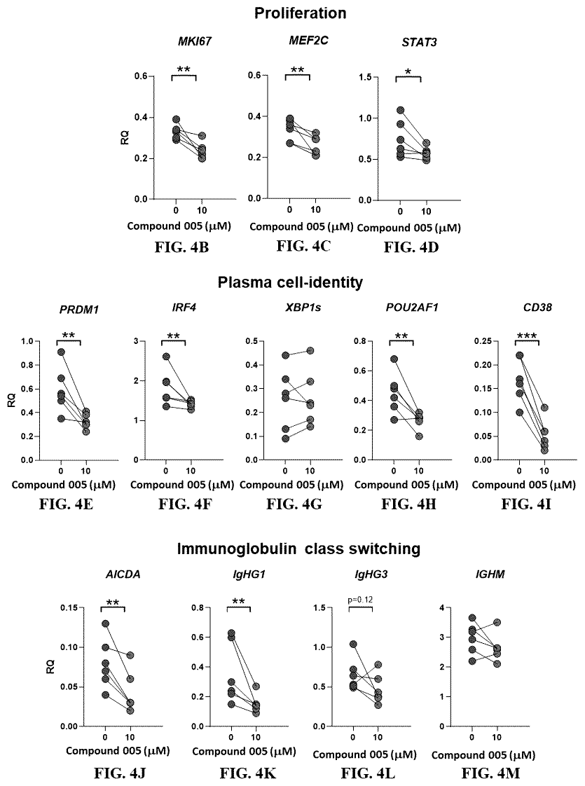

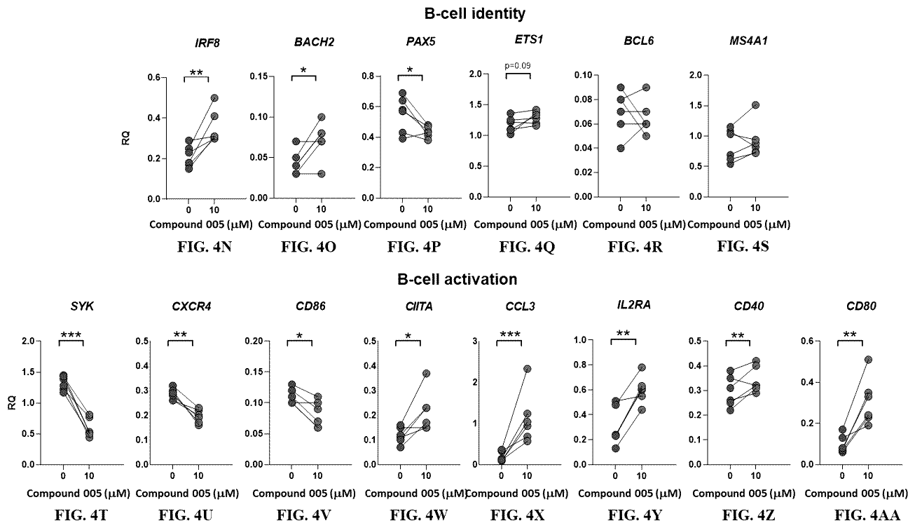

To evaluate the transcriptional program underlying phenotypical and functional changes during B cell differentiation mediated by compound 005 we assessed the expression of the panel of B-cell-related genes by high-throughput multiplex qPCR analysis. Separate clustering of day 4 and day 7 compound 005-treated and control-treated cells suggested a specific effect during two phases of the plasma cell differentiation model and the effect of the compound. Day 4 compound 005-treated B cells were most distant from other clusters suggesting the most substantial effect of targeting BOB1-OCT1 complex with the compound 005 during the activation phase plasma cell differentiation model (FIG. 4A). The clustering allows to identify gene modules linked to B-cell identity, plasma cell identity, and B cell activation, and revealed that plasma cell related genes were suppressed in B cells treated with compound 005. In contrast, genes related to the B cell identity and activation were upregulated. Notably downregulated were MK167, MEF2C, and STAT3 encoding for proteins essential for B cell proliferation (FIG. 4B-D), genes associated with plasma cell differentiation (PRDM1, IRF4, POU2AF1, CD38) (FIG. 4E-I) and isotype switching (AICDA, IgHG1) (FIG. 4J-M). In contrast, genes related to the B-cell identity including IRF8, BACH2 were upregulated (FIG. 4N-S).

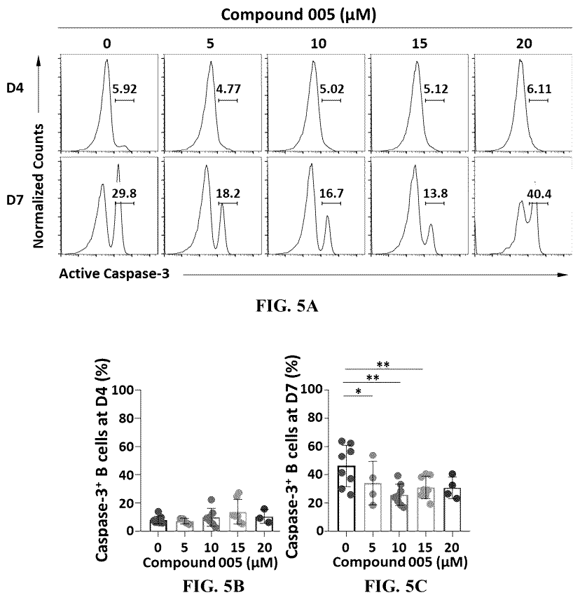

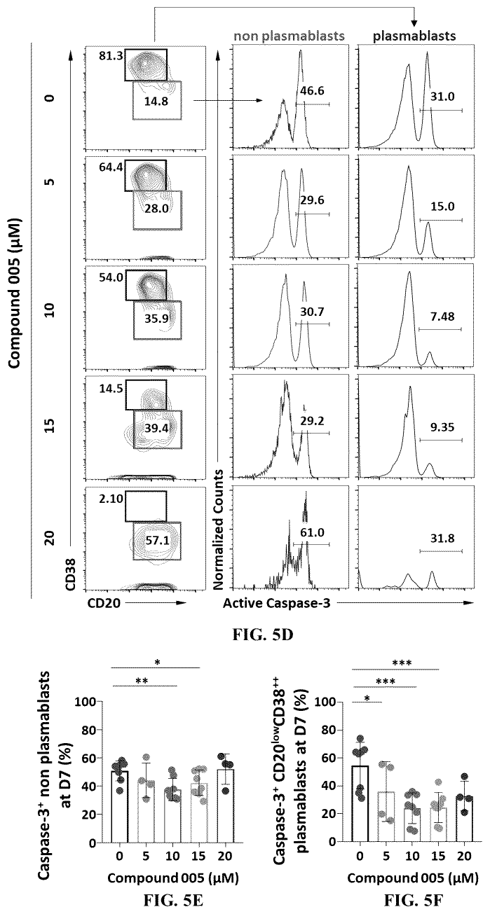

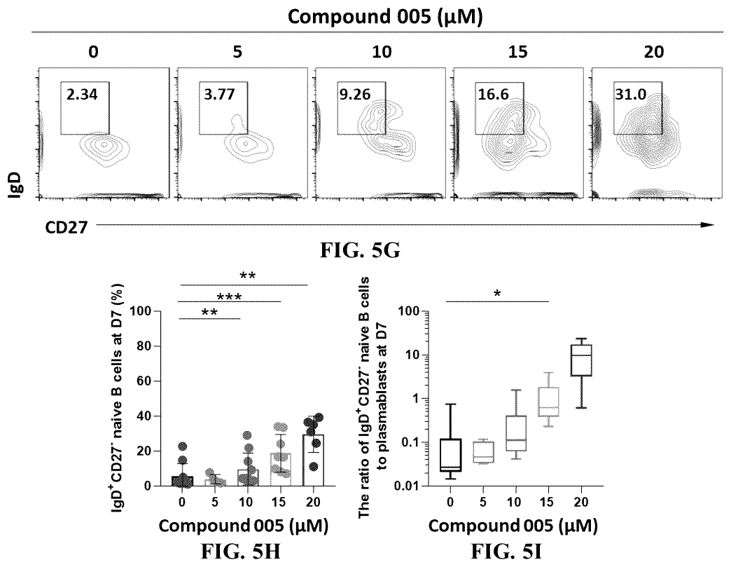

To evaluate whether the marked effect of BOB1 inhibition on the proliferation and the formation of CD20^{lo}CD38^{hi} cells was due to the induction of caspase-dependent apoptosis, we analyzed the percentage of caspase-3-positive B cells in the cultures. Concordant to what was previously reported, only a small proportion of control B cells (around 7%) showed caspase-3 positive staining during the activation phase (days 0-4). The presence of compound 005, even at high concentrations, did not affect the frequencies of caspase-3 positive cells (FIG. 5A). During the second phase, B cells stop proliferating and enter cell death apoptosis, leading to a significantly increased percentage of the caspase-3 positive population in the control condition (mean±SD of 7.8±2.7% at day 4 (FIG. 5B) versus 46.1±14.7% at day 7, P<0.0001 (FIG. 5C)). Surprisingly, the addition of compound 005 did not increase the frequency of caspase-3-positive B cells. Instead, cells cultured with 5, 10, and 15 μM of compound 005 showed a lower level of cell death apoptosis on day 7, as demonstrated by a significant decrease in the percentage of caspase-3 positive B cells (mean±SD of 33.9±15.5% (P=0.027), 25.6±7.5% (P=0.0002), and 30.8±7.9% (P=0.0005) for 5, 10, and 15 μM of compound 005, respectively (FIG. 5C). Detailed analysis revealed that inhibition of BOB1 reduced susceptibility to apoptosis in both CD20^{lo}CD38^{hi} plasmablast and CD20⁺CD38^{+/-} non-plasmablast populations (FIGS. 5D-F). Accordingly, the frequencies of naïve B cells were significantly increased with the increase in compound 005 concentration resulting in a substantially higher naïve B cell/plasmablast cell ratio (FIGS. 5G-I).

Collectively, these data indicate that the attenuating biological activity of BOB1-OCT1-DNA ternary complexes with compound 005 dose-dependently abrogates B-cell activation and differentiation into antibody-secreting plasma cells, the final effectors of the humoral immune response.

Compound 005 does not Affect the Regulatory Function of B Cells In Vitro

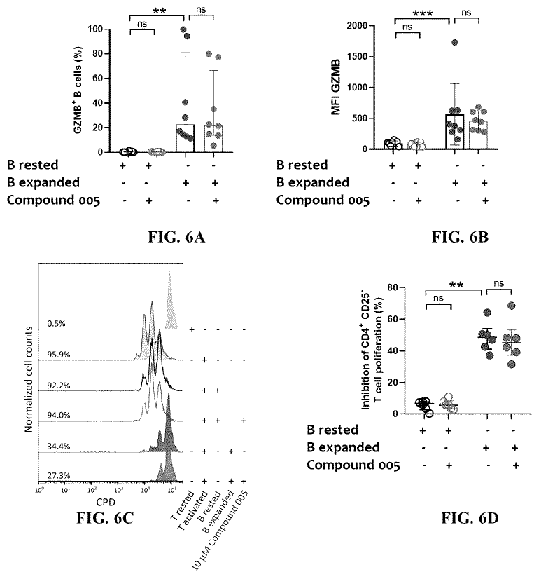

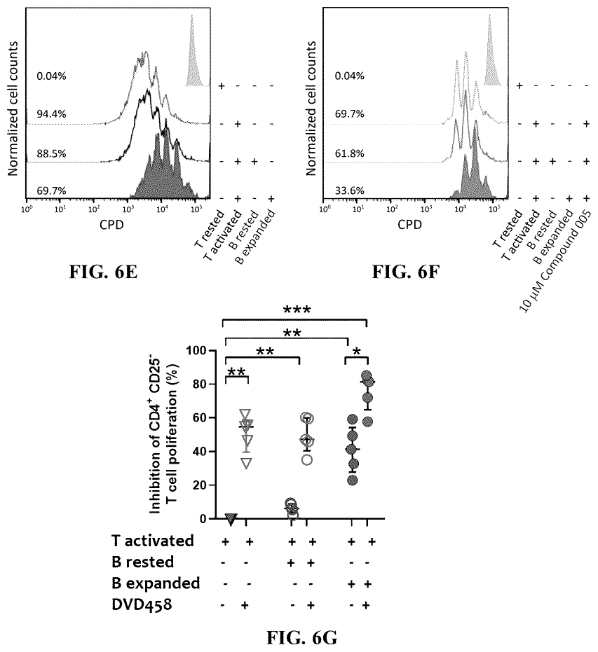

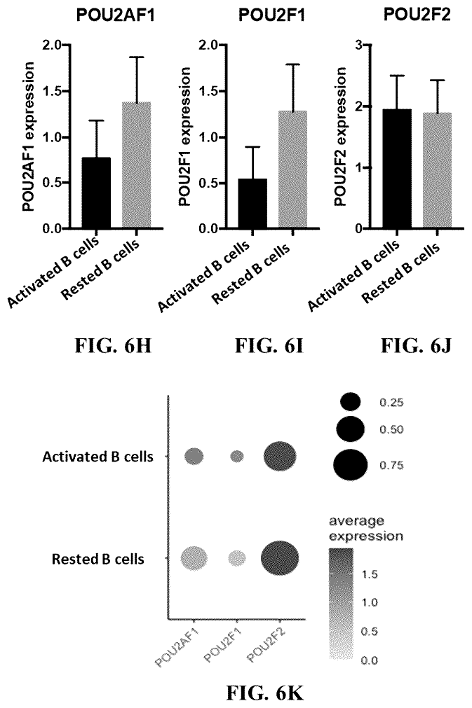

Different signals during T-cell-dependent activation determine whether B cells would differentiate into an effector of an immune response or a regulatory B cell with immunosuppressive properties. Therefore, we set out next to investigate whether targeting the BOB1-OCT(s) interaction by compound 005 could affect the regulatory function of B cells using a previously established in vitro model of expansion of human granzyme B (GZMB)-expressing B cells with regulatory properties. First of all, we evaluated whether compound 005 would impact the ability of B cells to express GZMB. To this end, we cultured human purified B cells in an expansion mixture containing F(ab′)2 anti-BCR, CpG ODN, CD40L, IL-2, and IL-21 (B expanded) for three days, including 24 hours of pre-expansion or left them unstimulated (B resting). 10 μM compound 005 (or 0.1% v/v DMSO for the vehicle control) was added daily to B-cell cultures. Flow cytometric analysis on day 3 revealed that B expanded express significantly more GZMB protein than B resting (FIGS. 6A-B). The presence of compound 005 did not affect GZMB expression (FIGS. 6A-B). Second, we demonstrated that B expanded that have been pre-stimulated for 24 hours with the expansion mixture are capable of inhibiting the proliferation of anti-CD3/CD28-activated autologous CD4⁺CD25⁻ effector T cells in contrast to B resting (48.8±9.8% versus 5.4±1.9%, P=0.002) (FIGS. 6C-D), consistent with previous reports. As shown in FIG. 6C and FIG. 6D, adding 10 μM of compound 005 during the 24-hours pre-stimulation phase did not affect the suppressive capacity of B expanded compared to the vehicle control. Third, we examined whether prolonged inhibition of BOB1-OCT(s) interaction would impact the suppressive capacity of B expanded by adding compound 005 (or 0.1% DMSO) during the 24-hours pre-stimulation phase and afterwards daily to the cultures. Analysis of T-cell proliferation revealed that B expanded but not B resting efficiently suppressed T-cell proliferation (48.8±9.8% versus 5.9±3.7%, P=0.002) (FIGS. 6E-G), similar to the previous experiment. Adding compound 005 inhibitor at 10 μM to T cells only or to the co-cultures of T cells and B resting reduced T-cell proliferation to approximately 50% (53.7±5.8% and 52.1±7.1%, respectively), indicating that inhibition of BOB1-OCT(s) interaction directly restricts T-cell proliferation (FIGS. 6E-G). Consistent with these data, adding 10 μM of compound 005 to the co-cultures of T cells and B expanded resulted in stronger suppression of effector T-cell proliferation than in the co-cultures with expanded B cells alone (79.7±4.9 versus 40.2±15.5%, P=0.01) (FIGS. 6E-G).

To address the question of why targeting the BOB1-OCT(s) interaction with compound 005 inhibits the differentiation of B cells into antibody-secreting plasmablasts but does not affect their ability to become GZMB-secreting cells with regulatory properties, we investigated the expression of BOB1 and its OCTs partners in both in vitro models. In line with in vitro-inhibition data, demonstrating that BOB1 is required for plasma cell generation (FIG. 3), expression of POU2AF1 gene encoding BOB1 is increased during plasma cell differentiation (FIG. 4A). In sharp contrast, single-cell RNA sequencing analysis revealed that POU2AF1 expression is downregulated in expanded GZMB⁺ B cells with regulatory functions (FIGS. 6H-K). These data further support the notion that BOB1 is not instrumental in Bregs, and, therefore, targeting the BOB1-OCT(s) interaction does not alter the suppressive potential of B cells.

### Compound 005 Inhibits Dose-Dependently T-Cell Proliferation and Selectively Suppresses Memory Th17 Responses

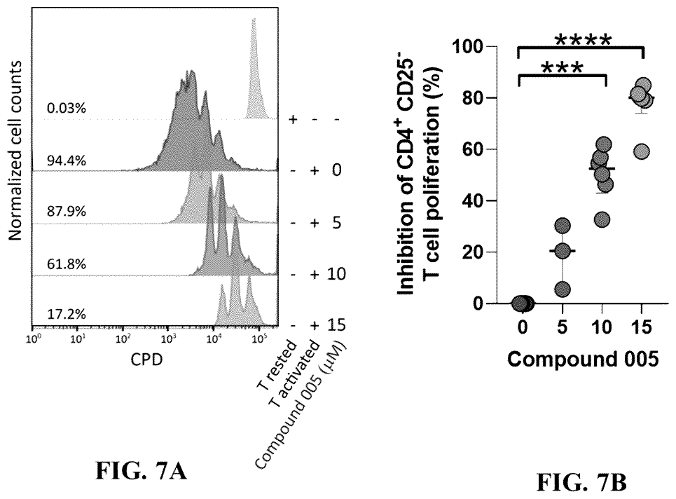

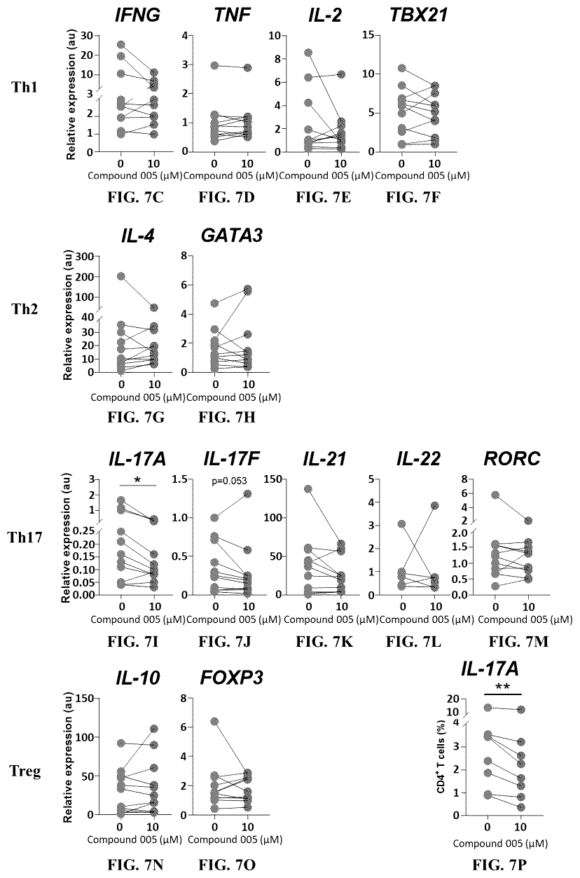

To confirm and extend the observation that targeting the BOB1-OCT(s) interaction suppresses effector T cell proliferation efficiently, we cultured CD4⁺CD25⁻ T cells in the presence of anti-CD3/CD28 microbeads and different concentrations of compound 005 for 3 days. We found that compound 005 suppresses anti-CD3/CD28-induced T-cell proliferation dose-dependently, resulting in 18.8±12.5% (P=0.18), 50.5±10.2% (P=0.002), and 77.5±9.2% (P<0.0001) inhibition for 5, 10, and 15 μM of compound 005 respectively (FIGS. 7A-B). OCT1 is constitutively expressed in T cells, whereas BOB1 is induced only after activation. It has been demonstrated that both proteins are selectively required for the generation of CD4 memory T cells and expression of multiple genes, including Th1, Th2 and Th17 type cytokines, upon antigen re-encounter in mice. Therefore, we next determined the consequences of inhibition of the OCT1-BOB1 complex on the expression of Th1-, Th2-, and Th17-associated cytokines during primary and secondary (memory) T-cell responses in vitro (FIGS. 7C-O). Data analysis revealed that re-stimulated T cells displayed a substantial increase in expression of IFNG (FIG. 7C), IL-17A (FIG. 7I), IL-17F (FIG. 7J), IL-22 (FIG. 7L) and IL-10 (FIG. 7N) cytokine-encoding genes compared to primary stimulated cells (data not shown). After initial activation (6 hours-stimulated cells) effect of compound 005 was observed only on TNF expression, which significant decrease was seen in the presence of a small molecule inhibitor (data not shown). As to the re-stimulated cells, treatment with compound 005 induced a significant reduction in IL-17A (median percentage decrease 43%, P=0.002) and IL-17F (median percentage decrease 27%, P=0.004), but not other cytokines and transcription factors mRNA level, including expression of Th-17 lineage-specific transcription factor RORC (FIG. 7M). Next, we investigated if BOB1-OCT1 inhibition also reduces IL-17A production on the protein level. In line with qPCR data, compound 005 suppressed the expression of IL-17A by CD4⁺ T cells (FIG. 7P).

These data support the notion that targeting the BOB1-OCT1 complex by compound 005 elicits a selective biological response by suppressing memory Th17 responses in vitro.

1-15. (canceled)

16. A compound of formula (I) 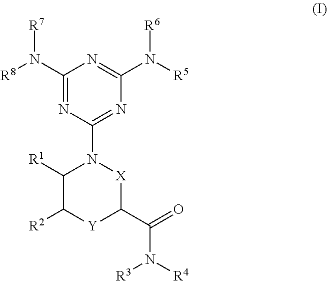
or a pharmaceutically acceptable salt and/or solvate thereof,
wherein:
X represents —(CH₂)ₙ—, wherein n is 0 or 1;
Y represents —O— or —(CH₂)ₘ—, wherein m is 1 or 2;
R¹ and R² each represents H; or R¹ and R² form together with the heterocycle to which they are attached a fused phenyl;
R³ and R⁴ each independently represents H, alkyl, —(CH₂)ₚ—NR⁹R¹⁰, aryl, or arylalkyl, wherein p is an integer ranging from 1 to 5,
wherein R⁹ and R¹⁰ each independently represent H or (C₁-C₄) alkyl; or R⁹ and R¹⁰ form together with the nitrogen atom to which they are attached a heterocycloalkyl;
wherein said aryl or arylalkyl is optionally substituted by at least one halogen, (C₁-C₄) alkyl, (C₁-C₄) haloalkyl, COOH, OH, or (C₁-C₄) alkoxy;
or R³ and R⁴ form together with the nitrogen atom to which they are attached a heterocycloalkyl;
wherein said heterocycloalkyl is optionally substituted by at least one aryl or arylalkyl;
wherein said aryl or arylalkyl is optionally substituted by at least one halogen, (C₁-C₄) alkyl, (C₁-C₄) haloalkyl, COOH, OH, or (C₁-C₄) alkoxy;
R⁵ represents aryl or arylalkyl;
wherein said aryl or arylalkyl is optionally substituted by at least one halogen, (C₁-C₄) alkyl, (C₁-C₄) haloalkyl, COOH, OH, or (C₁-C₄) alkoxy;
R⁶ represents H or (C₁-C₄) alkyl; and
R⁷ and R⁸ each independently represents H or alkyl substituted by at least one COOH or OH.

17. The compound according to claim 16, wherein R³ and R⁴ each independently represents H, alkyl, or —(CH₂)ₚ—NR⁹R¹⁰; wherein p is an integer ranging from 1 to 5, and wherein R⁹ and R¹⁰ form together with the nitrogen atom to which they are attached a heterocycloalkyl.

18. The compound according to claim 17, wherein R⁹ and R¹⁰ form together with the nitrogen atom to which they are attached a pyrrolidinyl.

19. The compound according to claim 16, wherein R³ represents aryl, wherein said aryl is optionally substituted by at least one halogen, (C₁-C₄) alkyl, (C₁-C₄) haloalkyl, COOH, OH, or (C₁-C₄) alkoxy; and R⁴ represents H.

20. The compound according to claim 16, wherein R³ and R⁴ form together with the nitrogen atom to which they are attached a heterocycloalkyl, wherein said heterocycloalkyl is optionally substituted by at least one aryl or arylalkyl.

21. The compound according to claim 16, wherein R³ and R⁴ form together with the nitrogen atom to which they are attached a heterocycloalkyl, wherein said heterocycloalkyl is optionally substituted by at least one phenyl or benzyl.

22. The compound according to claim 16, wherein R³ and R⁴ form together with the nitrogen atom to which they are attached a heterocycloalkyl, wherein said heterocycloalkyl is optionally substituted by at least one benzyl.

23. The compound according to claim 16, wherein R⁵ represents arylalkyl, wherein said arylalkyl is optionally substituted by at least one halogen, (C₁-C₄) alkyl, (C₁-C₄) haloalkyl, COOH, OH, or (C₁-C₄) alkoxy.

24. The compound according to claim 23, wherein R⁵ represents arylalkyl, wherein said arylalkyl is optionally substituted by at least one F.

25. The compound according to claim 16, wherein R⁶ represents H.

26. The compound according to claim 16, wherein R⁷ and R⁸ each independently represents alkyl substituted by at least one OH.

27. The compound according to claim 16, wherein R⁷ represents alkyl substituted by at least one COOH and R⁸ represents H.

28. The compound according to claim 16, wherein said formula (I) is of formula (I-A) 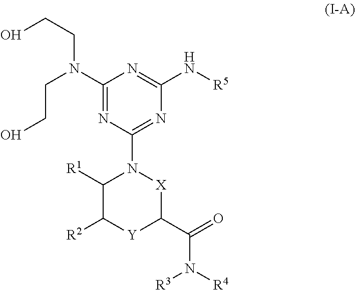
wherein
R¹, R², R³, and R⁴ are as defined in claim 1; and
R⁵ represents phenyl-(CH₂)_{q}—, wherein q is an integer ranging from 1 to 5, and wherein said phenyl is optionally substituted by at least one Br, Cl, F, methyl, or CF₃.

29. The compound according to claim 16, wherein said formula (I) is of formula (I-B) 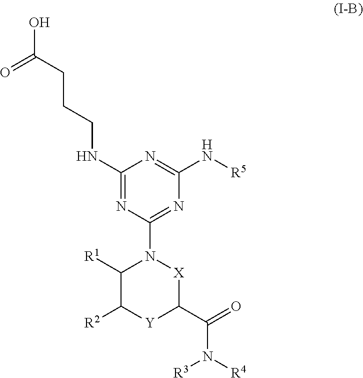
wherein
R¹, R², R³, and R⁴ are as defined in claim 1; and
R⁵ represents phenyl-(CH₂)_{q}—, wherein q is an integer ranging from 1 to 5, and wherein said phenyl is optionally substituted by at least one Br, Cl, F, methyl, or CF₃.

30. The compound according to claim 16, wherein said compound is selected from:

| | |
|---|---|
| 001 | (R)-1-(4-(bis(2-hydroxyethyl)amino)-6-((3- |
| | fluorobenzyl)amino)-1,3,5-triazin-2-yl)-N,N- |
| | diethylpyrrolidine-2-carboxamide |
| 002 | 1-(4-(bis(2-hydroxyethyl)amino)-6-((3- |
| | fluorobenzyl)amino)-1,3,5-triazin-2-yl)-N-(3-(pyrrolidin-1- |
| | yl)propyl)piperidine-3-carboxamide |
| 003 | (1-(4-(bis(2-hydroxyethyl)amino)-6-((3- |
| | fluorobenzyl)amino)-1,3,5-triazin-2-yl)piperidin-2- |
| | yl)(morpholino)methanone |
| 004 | 1-(4-(bis(2-hydroxyethyl)amino)-6-((3- |
| | fluorobenzyl)amino)-1,3,5-triazin-2-yl)-N-(4-bromo-3- |
| | (trifluoromethyl)phenyl)piperidine-2-carboxamide |
| 005 | 3-((4-(2-((3-chloro-4-methylphenyl)carbamoyl)-2,3- |
| | dihydro-4H-benzo[b][1,4]oxazin-4-yl)-6-((3-(4- |
| | fluorophenyl)propyl)amino)-1,3,5-triazin-2- |
| | yl)amino)propanoic acid |
| 006 | 3-((4-(3-(4-benzylpiperidine-1-carbonyl)piperidin-1-yl)- |
| | 6-((3-(4-fluorophenyl)propyl)amino)-1,3,5-triazin-2- |
| | yl)amino)propanoic acid |

and pharmaceutically acceptable salts and/or solvates thereof.

31. A pharmaceutical composition comprising the compound according to claim 16 and at least one pharmaceutically acceptable carrier.

32. A method for treating a disease in a subject in need thereof, said method comprising administering to the subject the compound according to claim 21, wherein said disease is selected from autoimmune diseases, transplanted organ rejection, graft-versus-host disease, and BOB1-related diseases.

33. The method according to claim 32, wherein the disease is selected from rheumatoid arthritis, type 1 diabetes, multiple sclerosis, primary biliary cirrhosis, end-stage chronic respiratory diseases (such as, for example, chronic obstructive pulmonary disease (COPD), cystic fibrosis (CF) and pulmonary hypertension (PAH)), germinal center-derived lymphomas, and Waldenstrom macroglobulinemia.

34. A process for manufacturing a compound according to claim 16, wherein said process comprises:
(a) a step of reacting cyanuric chloride of formula 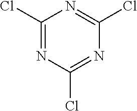
with the intracyclic NH secondary amine function of a compound of formula (A) 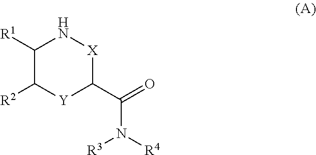
in presence of a base,
thereby obtaining a monosubstituted 1,3,5-triazine of formula (B) 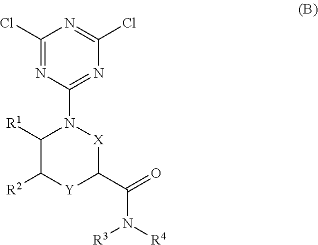
(b) a step of reacting said compound (B) with an amine of formula NHR⁵R⁶ in presence of a base, thereby obtaining a disubstituted 1,3,5-triazine; then
(c) a step of reacting the resulting disubstituted 1,3,5-triazine with an amine of formula NHR⁷R⁸ in presence of a base, thereby obtaining a trisubstituted 1,3,5-triazine;
or
(b) a step of reacting said compound (B) with an amine of formula NHR⁷R⁸ in presence of a base, thereby obtaining a disubstituted 1,3,5-triazine; then
(c) a step of reacting the resulting disubstituted 1,3,5-triazine with an amine of formula NHR⁵R⁶ in presence of a base, thereby obtaining a trisubstituted 1,3,5-triazine; and
(d) optionally, at least one step of deprotection of at least one alcohol, carboxylic acid and/or amine group present in the resulting trisubstituted 1,3,5-triazine;
thereby obtaining the compound of formula (I) or the pharmaceutically acceptable salt and/or solvate thereof.
